(19) United States
(12) Patent Application Publication
(10) Pub. No.: US 2025/0140417 A1
(43) Pub. Date: May 1, 2025

(54) METHODS FOR DETERMINING SECONDARY IMMUNODEFICIENCY

(71) Applicant: Immunosis Pty Ltd, Victoria (AU)

(72) Inventors: Simon Green, Victoria (AU); Benjamin Cocks, Victoria (AU); Emma Gail, Victoria (AU)

(21) Appl. No.: 18/683,191

(22) PCT Filed: Aug. 11, 2022
(86) PCT No.: PCT/AU2022/050881

§ 371 Date: Feb. 12, 2024

(30) Foreign Application Priority Data
Aug. 11, 2021 (AU) 2021902493

(51) Int. Cl. G16H 50/30; C12Q 1/6827; C12Q 1/6869; G06N 20/00; G16B 25/10; G16H 50/20; G16H 50/50

(52) U.S. Cl. CPC G16H 50/30; C12Q 1/6827; C12Q 1/6869; G06N 20/00; G16B 25/10; G16H 50/20; G16H 50/50; C12Q 2600/106; C12Q 2600/118; C12Q 2600/156; C12Q 2600/158

(57) ABSTRACT

The invention relates to a method for determining whether a subject has or is susceptible to developing a secondary immunodeficiency (SID), the method comprising using a linear mixed model to fit a transcriptome profile of the subject to a SID prediction equation developed by fitting into a linear mixed model a transcriptomic relationship matrix generated from a reference set of transcriptome profiles of reference subjects with and/or without SID, wherein the prediction equation's result indicates whether the subject has or is susceptible to SID. The invention relates to a method for developing a secondary immunodeficiency (SID) prediction equation for determining whether a subject has or is susceptible to developing a SID, the method comprising fitting into a linear mixed model a transcriptomic relationship matrix generated from a reference set of transcriptome profiles of reference subjects with and/or without SID to develop the SID prediction equation.

### RELATED APPLICATION

This application claims the benefit of priority to Australian provisional application no. 2021902493 filed 11 Aug. 2021, the entire contents of which is incorporated herein by reference.

### FIELD OF INVENTION

The present invention relates to methods for determining whether a subject has or is susceptible to developing a secondary immunodeficiency (SID).

### BACKGROUND OF THE INVENTION

Secondary immunodeficiencies (SIDs) result from a variety of factors that can affect a host with an intrinsically normal immune system, including infectious agents, drugs, metabolic diseases, and environmental conditions. These deficiencies of immunity are clinically manifested by an increased frequency, or unusual complications, of common infections and occasionally by the occurrence of opportunistic infections.

The secondary immunodeficiencies have a wide spectrum of presentation, depending on the magnitude of the offending external condition and on the host susceptibility. For example, the immunodeficiency induced by the use of corticosteroids and other immunosuppressive drugs depends on the dose used and, to a lesser degree, on concomitant disease processes of the host, such as the presence of sepsis. AIDS, resulting from infection by HIV, is the best known secondary immunodeficiency largely because of its prevalence and its high mortality rate if not treated. However, the most common immunodeficiency worldwide results from severe malnutrition, affecting both innate and adaptive immunity. Other causes of SIDs include and are not limited to: diabetes, B-cell or T-cell malignancies and spleen removal. The treatment in secondary immunodeficiencies is generally achieved with the management of the primary condition or the removal of the causative agent or drug. Such agents and drugs can include and not limited to immunosuppressants used in organ transplants and chemotherapy-based cancer treatments. Treatment of SID can also be with polyvalent immunoglobulin, vaccination or antibiotic prophylaxis.

Owing to the variety of clinical scenarios causing SID, SID is usually indicated on the basis of increased frequency or severity of infections. The current diagnostic procedures involve a myriad of specialized, costly and laborious functional tests including lymphocyte proliferation and cytotoxicity assays, flow cytometry, measurement of serum immunoglobulin levels, complete blood cell counts, neutrophil function tests, and complement assays.

The specific immune defects and clinical presentation in SIDs affect both the innate and the adaptive immunity. Gene expression analysis of the innate cells is not used or thought of as a direct diagnosis approach for SID, with current approaches for diagnosis relying on cell-based functional information on the composition and performance of the immune system. Functional insight gained from gene expression analysis may allow the identification of sets of genes whose expression is indicative of, and can discriminate, SID from immune competent individuals including individuals with other disorders of the immune system.

Importantly, a comprehensive analysis of gene expression levels in patients as a complex phenotype analysis has not been used or thought of as a direct diagnosis approach for SID.

The risk of SIDs can be influenced by genetic defects that do not primarily affect the immune system (as in the case of primary immunodeficiency (PID)), but they can present with impaired immunity to infections resulting from metabolic and cellular dysfunction, such as poor expression of adhesion molecules or defects in the DNA repair machinery. Genetic syndromes such as Down syndrome, Turner syndrome, Cushing's syndrome, and cystic fibrosis all increase the risk of infection and suggest a complex interaction between gene defects and immune function. Also, in some cases less severe mutations in PID genes, or mutations when heterozygous that do not lead to PID such as in the TACI gene may predispose patients to SID or more severe SID.

Diagnosing the active SID, and predicting SID susceptibility are both required in clinical settings. For example low antibody levels may be indicative of active SID or a surrogate for predicting likelihood a patient will develop SID with treatment or disease.

Next-generation sequencing (NGS), including whole genome sequencing (WGS) or whole exome sequencing (WES), has made it possible to simultaneously amplify and sequence millions of DNA fragments from a single subject within a few days. However, identification of causative mutations can be challenging because there are many nucleotide variants to measure and new variants that are detected by NGS are difficult to interpret since they often relate to poorly characterized genes or have unpredictable biological consequences on protein function.

A recognized limitation of DNA sequencing is that it does not provide functional information on the performance of the immune system, key information that is also required for SID diagnosis.

As noted above, a defining characteristic of SID is recurrent infection, owing to the inability of the immune system to manage microbial colonisation and invasion. While identification of specific pathogens may be useful and inform treatment in some cases, monitoring commensal microbial community composition may also provide useful information for managing SID. Microbial community is increasingly being shown to have a functional interaction with the immune system [2], including in the skin of primary immunodeficiency patients [3], a group of diseases with similar clinical manifestation to SID.

There exists a need for an efficient and accurate diagnostic method for SID, which can be deployed reliably in a clinical setting.

Reference to any prior art in the specification is not an acknowledgement or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

The inventors provide a method of determining whether a subject has or is susceptible to developing SID. The method comprises RNA analyses (RNAseq) of gene expression, i.e. the transcriptome, and optionally gene sequence mutations, and further comprises using a linear mixed model with RNA expression levels as input (not sequence or SNPs) to detect lack of function in the immune system reflected in the transcriptome, and optionally detection of specific SID sequence mutations. In addition, the inventors provide a method of using metagenome profiling as a measure of commensal microbial community structure in combination with RNA-sequencing mixed model analysis to determine whether a subject has or is susceptible to developing SID.

Accordingly, in one aspect the present invention provides a method for determining whether a subject has or is susceptible to developing a secondary immunodeficiency (SID), the method comprising:
using a linear mixed model to fit a transcriptome profile of the subject to a SID prediction equation developed by fitting into a linear mixed model a transcriptomic relationship matrix generated from a reference set of transcriptome profiles of reference subjects with and without SID,

wherein the prediction equation's result indicates whether the subject has or is susceptible to SID.

In another aspect the present invention provides a method for determining whether a subject has or is susceptible to developing a secondary immunodeficiency (SID), the method comprising:
generating a transcriptome profile from a sample from the subject; and
using a linear mixed model to fit the transcriptome profile of the subject to a SID prediction equation developed by fitting into a linear mixed model a transcriptomic relationship matrix generated from a reference set of transcriptome profiles of reference subjects with and without SID,

wherein the prediction equation's result indicates whether the subject has or is susceptible to SID.

In a further aspect the present invention provides a method for determining whether a subject has or is susceptible to developing a secondary immunodeficiency (SID), the method comprising:
obtaining a sample from a subject;
generating a transcriptome profile from the sample; and
using a linear mixed model to fit a transcriptome profile of the subject to a SID prediction equation developed by fitting into a linear mixed model a transcriptomic relationship matrix generated from a reference set of transcriptome profiles of reference subjects with and without SID,

wherein the prediction equation's result indicates whether the subject has or is susceptible to SID.

In one aspect, the present invention provides a method for developing a secondary immunodeficiency (SID) prediction equation for determining whether a subject has or is susceptible to developing a SID, the method comprising:
fitting into a linear mixed model a transcriptomic relationship matrix generated from a reference set of transcriptome profiles of reference subjects with and without SID to develop the SID prediction equation.

In another aspect, the present invention provides a method for developing a secondary immunodeficiency (SID) prediction equation for determining whether a subject has or is susceptible to developing a SID, the method comprising:
generating a reference transcriptome profile from reference subjects;
generating a reference set of transcriptome profiles; and
fitting into a linear mixed model a transcriptomic relationship matrix generated from the reference set of transcriptome profiles of reference subjects with and without SID to develop the SID prediction equation.

In another aspect, the present invention provides a method for developing a secondary immunodeficiency (SID) prediction equation for determining whether a subject has or is susceptible to developing a SID, the method comprising:
obtaining sample(s) from one or more subjects with and without SID;
generating a reference transcriptome profile from each subject;
generating a reference set of transcriptome profiles; and
fitting into a linear mixed model a transcriptomic relationship matrix generated from the reference set of transcriptome profiles of reference subjects with and without SID to develop the SID prediction equation.

In any embodiment of the above methods, the method further comprises measuring or determining the transcriptome profile of the subject for whom the determination of SID or susceptibility to SID is to be made.

In one aspect, the present invention provides a method for longitudinal monitoring of SID in a subject, the method comprising:
using a linear mixed model to evaluate the transcriptome profiles of a subject's repeat samples by a SID prediction equation developed by fitting into a linear mixed model a transcriptomic relationship matrix generated from the reference transcriptome profile(s) with and without SID,

wherein the prediction equation's result indicates whether the subject has a change in SID status.

In any aspect or embodiment relating to longitudinal monitoring of SID in a subject, the SID prediction equation is developed as described herein.

In another aspect, the present invention provides a method for longitudinal monitoring of SID in a subject, the method comprising:
generating a transcriptome profile from the obtained samples from a subject, wherein the samples are obtained at different times; and
using a linear mixed model to evaluate a transcriptome profile of the subject's repeat, further or subsequent samples, to a SID prediction equation developed by fitting into a linear mixed model a transcriptomic relationship matrix generated from the reference transcriptome profiles with and without SID,

wherein the prediction equation's result indicates whether the subject has a change in SID status.

In another aspect, the present invention provides a method for longitudinal monitoring of SID in a subject, the method comprising:
obtaining a reference sample from a subject;
obtaining a subsequent sample from the subject at a later time to the reference sample;
generating a reference transcriptome profile from the reference sample and a transcriptome profile from the subsequent sample; and
using a linear mixed model to evaluate a transcriptome profile of the subject's subsequent sample to a SID prediction equation developed by fitting into a linear mixed model a transcriptomic relationship matrix generated from the reference transcriptome profile of the subject at the start of monitoring,
   wherein the prediction equation's result indicates whether the subject has a change in SID status.

In one aspect, the present invention provides a method for developing a secondary immunodeficiency (SID) prediction equation for longitudinal monitoring of SID in a subject, the method comprising:
fitting into a linear mixed model a transcriptomic relationship matrix generated from a reference transcriptome profile of a subject at the start of monitoring to establish a baseline reference SID equation evaluation or score.

In another aspect, the present invention provides a method for developing a secondary immunodeficiency (SID) prediction equation suited for longitudinal monitoring of SID in a subject, the method comprising:
generating a reference transcriptome profile from reference subjects;
generating a reference set of transcriptome profiles from the reference subjects; and
fitting into a linear mixed model a transcriptomic relationship matrix generated from a reference transcriptome profiles of the subjects at the start of monitoring to develop the SID equation;
using a linear mixed model to evaluate the transcriptome profiles of a subject's repeat samples by a SID prediction equation developed by fitting into a linear mixed model a transcriptomic relationship matrix generated from longitudinal reference transcriptome profile(s).

In any embodiment, the reference set of transcriptome profiles and/or transcriptome profile of the subject for whom the determination of SID or susceptibility to SID is to be made includes at least 50, at least 100, at least 150, at least 200, at least 250, at least 300, at least 350, at least 400, at least 450, or all 500 of the genes listed in Table 1 Table 2, Table 3, Table 4, or FIG. 3, or Tables 1, 2, 3, 4 and FIG. 3.

In another aspect, the present invention provides a method comprising determining a transcriptome profile of a sample from a subject suspected of having or susceptible to a SID. The transcriptome profile including at least 50, at least 100, at least 150, at least 200, at least 250, at least 300, at least 350, at least 400, at least 450, or all 500 of the genes listed in Table 1, Table 2, Table 3, Table 4 or FIG. 3, or Tables 1, 2, 3, 4 and FIG. 3.

In a preferred embodiment of the invention, the linear mixed model is best linear unbiased prediction (BLUP), BayesR, or machine learning approaches. In a further embodiment of the invention, the machine learning approaches one of elastic net, ridge regression, lasso regression, random forest, gradient boosting machines, support vector machines, multilayer perceptrons (MLP) or convolutional neural networks (CNN).

In an embodiment of the invention, the SID prediction equation provides an absolute predictive score. In one embodiment, the absolute predictive score is greater than or equal to 0.1, greater than or equal to 0.2, greater than or equal to 0.3, greater than or equal to 0.4, greater than or equal to 0.5, greater than or equal to 0.6, greater than or equal to 0.7, greater than or equal to 0.8, or greater than or equal to 0.9, or about 0.1, about 0.2, about 0.3, about 0.4, about 0.5, about 0.6, about 0.7, about 0.8, or about 0.9. In another embodiment the absolute predictive score is from about 0.1 to about 0.9. In another embodiment, the absolute predictive score is greater than or equal to −0.3, greater than or equal to −0.2, greater than or equal to −0.1, greater than or equal to 0.0, greater than or equal to 0.1, greater than or equal to 0.2, greater than or equal to 0.3, or greater than or equal to 0.4, or about −0.3, about −0.2, about −0.1, about 0.0, about 0.1, about 0.2, about 0.3, or about 0.4. Preferably the absolute predictive score is from about −0.3 to about 0.4.

In an embodiment of the invention, the SID prediction equation provides a relative predictive score, wherein the relative score is calculated by subtracting the healthy control score (determined from a population of known healthy control subjects) from the patient score (determined from the sample of the subject to be diagnosed). In one embodiment, the relative predictive score is greater than 0, greater than 0.1 and greater than 0.2, or about 0, about 0.1 or about 0.2. In another embodiment the relative predictive score is from about 0 to about 0.2. In another embodiment, the relative predictive score is greater than −0.5, greater than −0.4 and greater than −0.3, or about −0.5, about −0.4 or about −0.3. Preferably the relative predictive score is from about −0.5 to −0.3,

In another embodiment of the invention, the SID prediction equation provides a relative predictive score, wherein the relative score is calculated by subtracting the reference control score (determined from the first sample of a subject) from the a repeated, further or subsequent sample score (determined from the any repeat, further or subsequent sample obtained from a subject). In one embodiment, a relative predictive score is greater than 0, greater than 0.1 and greater than 0.2, or about 0, about 0.1 or about 0.2. In another embodiment, the relative predictive score is greater than −0.5, greater than −0.4 and greater than −0.3, or about −0.5, about −0.4 or about −0.3. Preferably the relative predictive score is from about −0.5 to −0.3.

In any embodiment of the invention, the SID prediction equation further provides a read-out of a SID-associated gene mutation. Examples of SID mutations are known in the art, including those in the receptor for G-CSF (G-CSF-R), encoded by CSF3R, and described in Sprenkeler et al. Br J Haematology, 2020: 191(5): 930-934, or those described in Orru et al Cell 2013 Sep. 26; 155(1):242-56.

In any embodiment of the above methods, the reference set further comprises a RNA sequence mutation profile.

In any embodiment of the above methods, the method further comprises measuring or determining a RNA sequence mutation profile of the subject for whom the determination of SID or susceptibility to SID is to be made.

In any embodiment of the above methods, transcriptome profile is used to provide further information in relation to the defective pathway within the subject with SID. For example, a report may be generated that states the patient has a deficiency in the Fc receptor signalling pathway, the complement pathway or the Interferon signalling pathway. This provides information to the clinician that may assist with prescribing treatment options.

In preferred embodiments of the invention, the mutation profile comprises:
a) a RNA sequence of a gene comprising a known mutation associated with, involved in, or causative of, SID;
b) a new mutation, optionally a frameshift mutation or a missense amino acid changing mutation, or nonsense stop codon, that affects structure or function of a protein encoded by a known gene mutation associated with, involved in, or causative of, SID;
c) a dominant mutation in one allele that is associated with, involved in, or causative of, SID;
d) two different mutations in the same gene, but on two different alleles that are associated with, involved in, or causative of, SID;
e) a known mutation or SNP in RNA that is inferred or imputed by linkage to a co-occurring marker for a mutation associated with, involved in, or causative of, SID;
f) absence of expression of a gene normally expressed in non-SID subjects indicating a regulatory defect or destabilising mutation;
g) a defective exon structure indicating a splicing defect;
h) one or more, optionally one to three, additional mutations associated with, involved in, or causative of, SID; or
i) a sequence of more than one other gene, or an imputed sequence of more than one other gene, that associated with, involved in, or causative of, SID severity.

In any embodiment of the above methods, the reference set further comprises a DNA sequence mutation profile.

In any embodiment of the above methods, the method further comprises measuring or determining the DNA sequence mutation profile of the subject for whom the determination of SID or susceptibility to SID is to be made. Preferably, the linear mixed model is used to fit the transcriptome profile and the DNA sequence mutation profile of the subject to the SID prediction equation. In any embodiment of the above methods, the reference set further comprises a metagenome profile and the linear mixed model is used to fit the transcriptome profile and a metagenome profile of the subject to the SID prediction equation. Immune suppressive drugs used in transplantation or to treat autoimmunity can affect mucosal microbiome profiles, affecting specific bacteria and furthermore, the pre-transplantation salivary bacterial composition including specific species is associated with susceptibility to infection. The mucosal bacterial composition and specific species such as actinobacteria can be predictive using a machine learning approach for severity of graft vs host disease in stem cell transplantation

In a preferred embodiment of the above methods, the metagenome profile is obtained from a mouth swab, nose swab, throat swab, saliva, faeces, or skin.

In a further preferred embodiment, the subject is human.

In a further aspect of the present invention there is a method for determining whether a subject has or is susceptible to developing a secondary immunodeficiency (SID), the method comprising using a linear mixed model to fit a metagenomics profile of the subject to a SID prediction equation developed by fitting into a linear mixed model a metagenomic relationship matrix generated from a reference set of metagenomic profiles of reference subjects with and without SID, wherein the prediction equation's result indicates whether the subject has or is susceptible to SID.

In another aspect of the present invention, a method for longitudinal monitoring of SID in a subject, the method comprising using a linear mixed model to fit a metagenomics profile of the subject to a SID prediction equation developed by fitting into a linear mixed model a metagenomic relationship matrix generated from a reference set metagenomic profile of the subject for whom the monitoring of SID is to be made, wherein the prediction equation's result indicates whether the subject has a change in SID status.

It will be understood that the transcriptome profile or sequence mutation profile is obtained from sputum, blood, amniotic fluid, plasma, semen, bone marrow, tissue, urine, peritoneal fluid, or pleural fluid, optionally obtained by fine needle biopsy.

It will be further understood that the transcriptome profile or sequence (DNA and/or RNA) mutation profile is generated in vitro or ex vivo.

It will be further understood that the transcriptome profile or sequence (DNA and/or RNA) mutation profile is generated in vitro, ex vivo, or in-silico.

In some embodiments of the above methods, the method is not practised on a human or animal body.

In some embodiments of the above methods, the method excludes any set of direct data acquisition practised on the human or animal body.

In a preferred embodiment of the above methods, the blood comprises peripheral blood mononuclear cells.

In any aspect or embodiment, the transcriptome, sequence (DNA and/or RNA) mutation profile and metagenome profile is determine from a sample previously obtained from the subject.

In another aspect, the present invention provides a method for determining whether a subject has or is susceptible to developing a secondary immunodeficiency (SID), the method comprising using a linear mixed model to fit a transcriptome profile of the subject to a SID prediction equation developed by fitting into a linear mixed model a transcriptomic relationship matrix generated from a reference set of transcriptome profiles of reference subjects with and without SID, wherein the prediction equation's result indicates whether the subject has or is susceptible to SID.

In another aspect of the present invention, a method for longitudinal monitoring of SID in a subject, the method comprising using a linear mixed model to fit a transcriptome profile of the subject to a SID prediction equation developed by fitting into a linear mixed model a transcriptomic relationship matrix generated from a reference set transcriptomic profile of the subject for whom the monitoring of SID is to be made, wherein the prediction equation's result indicates whether the subject has a change in SID status.

In another aspect, the present invention provides a method of treating secondary immunodeficiency (SID) in a subject who has or is susceptible to developing a secondary immunodeficiency (SID), the method comprising:
determining whether the subject has or is susceptible SID by performing or having performed a method as described herein; and
wherein if the subject has or is susceptible SID then administering to the subject therapy specific to the SID.

In another aspect, the present invention provides a method of treating secondary immunodeficiency (SID) in a subject who has or is susceptible to developing a secondary immunodeficiency (SID), the method comprising:
determining whether the subject has SID by using a linear mixed model to fit a transcriptome profile of the subject to a SID prediction equation developed by fitting into a linear mixed model a transcriptomic relationship matrix generated from a reference set of transcriptome profiles of reference subjects with and without SID, wherein the prediction equation's result indicates whether the subject has or is susceptible to SID,

wherein if the subject has or is susceptible to developing a secondary immunodeficiency (SID) then administering to the subject therapy specific to the SID.

In another aspect, the present invention provides a use of the therapy specific to secondary immunodeficiency (SID) in the manufacture of a medicament for treating secondary immunodeficiency (SID) in a subject who has or is susceptible to developing a secondary immunodeficiency (SID), wherein the subject is diagnosed by the method as described herein.

In another aspect, the present invention provides a method of determining the efficacy of SID therapy in a subject, the method comprising:
providing a first sample obtained from a subject before receiving SID therapy;
providing a second sample obtained from a subject during, or after, receiving SID therapy;
using a linear mixed model to fit a transcriptome profile of the subject's first and second samples to a SID prediction equation developed by fitting into a linear mixed model a transcriptomic relationship matrix generated from a reference set of transcriptome profiles of reference subjects with and without SID,

wherein the change in transcriptome profile from the first and second samples indicates efficacy of the SID therapy in the subject.

In one embodiment of the above methods, the SID therapy is intravenous immunoglobulin (IVIG) administration. In a further embodiment, the intravenous immunoglobulin (IVIG) is administered at a dose of between 200-800 mg/kg. In a further embodiment, the dose of intravenous immunoglobulin (IVIG) is administered every 3-4 weeks.

In another embodiment of the above methods, the SID therapy is subcutaneous immunoglobulin (SCIG) administration. In a further embodiment, the subcutaneous (SCIG) is administered either daily, weekly or biweekly (every 2 weeks) at a dose that is calculated for each patient according to the manufacturer's instructions taking into account their immunoglobulin trough levels and previous dose of IVIG.

In another embodiment, the present invention provides a method for reducing, inhibiting or ameliorating secondary immunodeficiency (SID) caused by an agent used in the treatment for a subject, the method comprising:
determining whether the subject has SID by performing or having performed a method as described herein; and
wherein if the subject has SID then the amount, dose or frequency of administration of the agent is reduced.

Preferably, the agent is any one as described herein, for example a chemotherapy or immunosuppressive drug (such as when a subject is undergoing or has undergone a transplant, for example lung transplant).

In any embodiment of the above methods, the secondary immunodeficiency can be selected from any one of the following types: antibody deficiencies, combined immunodeficiencies, phagocytic cell deficiencies, immune dysregulation, or complement deficiencies. Preferably, the secondary immunodeficiency is an antibody deficiency.

In any embodiment of the above methods, the cause of secondary immunodeficiency can be selected the group consisting of: malnutrition; diabetes mellitus; chronic uremia; advanced age; genetic syndromes including but not limited to trisomy 21; anti-inflammatory, immunomodulatory and immunosuppressive drugs including but not limited to: corticosteroids, calcineurin inhibitors and cytotoxic drugs; surgery and trauma; environmental conditions not limited to: UV light, radiation and hypoxia; infectious diseases including but not limited to viral infection, such as HIV infection; or any other cause of secondary immunodeficiency described herein.

In another aspect subclinical SID, or future susceptibility to SID, can be detected or monitored longitudinally in individuals where their immune system health may be affected by lifestyle factors such as exercise, sickness, infection, stress, toxins, recreational drugs, medicines, fitness, weight, diet, pregnancy, supplements, programs, treatments, environment changes, probiotics, anti-inflammatories, antibiotics, glucocorticoids, gum disease, or surgery. For example, a measure for immune health (subclinical or predisposition to SID) could be whole transcriptome predictions of markers of immune health examples being serum or mucosal antibody levels such as levels of IgG1 or IgA1/IgA2 respectively.

In any aspect or embodiment herein relating to determining SID, the methods or uses may be performed to determine or diagnose subclinical SID.

In one aspect the present invention provides a computer-implemented method for processing genomic information, the genomic information comprising a subject transcriptome profile, the method comprising:
accessing a reference set of transcriptome profiles of reference subjects, each reference subject either having or not having a secondary immunodeficiency (SID);
generating a transcriptomic relationship matrix from the reference set of transcriptome profiles;
fitting the transcriptomic relationship matrix into a linear mixed model to generate a SID prediction equation; and
fitting the subject transcriptome profile to the SID prediction equation.

In another aspect the present invention provides a computer-implemented method for generating a secondary immunodeficiency (SID) prediction equation, the method comprising:
accessing a reference set of transcriptome profiles of reference subjects, each reference subject either having or not having a secondary immunodeficiency (SID);
generating a transcriptomic relationship matrix from the reference set of transcriptome profiles; and
fitting the transcriptomic relationship matrix into a linear mixed model to generate the SID prediction equation.

In any embodiment of the above methods, further comprises measuring or determining the transcriptome profile of the subject for whom the determination of SID or susceptibility to SID is to be made.

In another embodiment of the computer-implemented method for processing genomic information, the genomic information comprising a subject transcriptome profile, the method comprising:
accessing a reference set transcriptome profile of a subject for whom longitudinal monitoring of SID is to be made;
generating a transcriptomic relationship matrix from the reference set transcriptome profile;
fitting the transcriptomic relationship matrix into a linear mixed model to generate a SID prediction equation; and
fitting the subject transcriptome profile to the SID prediction equation.

In another aspect of the computer-implemented method for generating a secondary immunodeficiency (SID) prediction equation, the method comprising:
accessing a reference set transcriptome profile of the subject for whom longitudinal monitoring of SID is to be made;
generating a transcriptomic relationship matrix from the reference set transcriptome profile; and
fitting the transcriptomic relationship matrix into a linear mixed model to generate the SID prediction equation.

In preferred embodiment of the invention, the linear mixed model is best linear unbiased prediction (BLUP), BayesR, random forest or machine learning approaches, including those as defined herein.

In any embodiment of the above methods, the reference set further comprises a RNA sequence mutation profile.

In any embodiment of the above methods, the method further comprises measuring or determining a RNA sequence mutation profile of the subject for whom the determination of SID or susceptibility to SID is to be made.

In any embodiment of the above methods, the reference set further comprises a DNA sequence mutation profile.

In any embodiment of the above methods, the method further comprises measuring or determining the DNA sequence mutation profile in the subject for whom the determination of SID or susceptibility to SID is to be made. Preferably, the linear mixed model is used to fit the transcriptome profile and the DNA sequence mutation profile of the subject to the SID prediction equation.

In any embodiment of the above methods, the reference set further comprises a metagenome profile and the linear mixed model is used to fit the transcriptome profile and a metagenome profile of the subject to the SID prediction equation.

In a further aspect of the present invention a method determining whether a subject has or is susceptible to developing a secondary immunodeficiency (SID), the method comprising using a linear mixed model to fit a metagenomics profile of the subject to a SID prediction equation developed by fitting into a linear mixed model a metagenomic relationship matrix generated from a reference set of metagenomic profiles of reference subjects with and without SID, wherein the prediction equation's result indicates whether the subject has or is susceptible to SID.

In any aspect or embodiment, the subject does not have primary immunodeficiency (PID), or is not suspected of having PID.

In another aspect the present invention provides a non-transitory computer-readable medium storing instructions, which when executed by a processor cause the processor to:
access a reference set of transcriptome profiles of reference subjects, each reference subject either having or not having a secondary immunodeficiency (SID);
generate a transcriptomic relationship matrix from the reference set of transcriptome profiles;
fit the transcriptomic relationship matrix into a linear mixed model to generate a SID prediction equation;
receive a subject transcriptome profile; and
fit the subject transcriptome profile to the SID prediction equation.

In another aspect the present invention provides a non-transitory computer-readable medium storing instructions, which when executed by a processor cause the processor to:
access a reference set of transcriptome profiles of reference subjects, each reference subject either having or not having a secondary immunodeficiency (SID);
generate a transcriptomic relationship matrix from the reference set of transcriptome profiles; and
fit the transcriptomic relationship matrix into a linear mixed model to generate the SID prediction equation.

In another aspect, the present invention provides a method for longitudinal monitoring of SID in a subject, the method comprising using a linear mixed model to fit a metagenomics profile of the subject to a SID prediction equation developed by fitting into a linear mixed model a metagenomic relationship matrix generated from a reference set metagenomic profile of the subject for whom the monitoring of SID is to be made, wherein the prediction equation's result indicates whether the subject has a change in SID status.

In another embodiment, the present invention provides a non-transitory computer-readable medium storing instructions, which when executed by a processor cause the processor to:
access a reference set transcriptome profile of the subject for whom the longitudinal monitoring of SID is to be made;
generate a transcriptomic relationship matrix from the reference set transcriptome profile;
fit the transcriptomic relationship matrix into a linear mixed model to generate a SID prediction equation;
receive the subject transcriptome profile; and
fit the subject transcriptome profile to the SID prediction equation.

In a further aspect the present invention provides a non-transitory computer-readable medium storing instructions, which when executed by a processor cause the processor to:
access a reference set transcriptome profile of the subject for whom the longitudinal monitoring of SID is to be made;
generate a transcriptomic relationship matrix from the reference set transcriptome profile; and
fit the transcriptomic relationship matrix into a linear mixed model to generate the SID prediction equation.

In preferred embodiment of the invention, the linear mixed model is best linear unbiased prediction (BLUP), BayesR, or machine learning approaches, including those as defined herein. In a further embodiment of the invention, the machine learning approaches are one of elastic net, ridge regression, lasso regression, random forest, gradient boosting machines, support vector machines, multilayer perceptrons (MLP) or convolutional neural networks (CNN).

In any embodiment of the above a non-transitory computer-readable medium storing instructions, the reference set further comprises a RNA sequence mutation profile.

In any embodiment of the above a non-transitory computer-readable medium storing instructions, the reference set further comprises a DNA sequence mutation profile.

In any embodiment of the above a non-transitory computer-readable medium storing instructions, the reference set further comprises a metagenome profile and the linear mixed model is used to fit the transcriptome profile and a metagenome profile of the subject to the SID prediction equation.

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF DRAWINGS

FIG. 1. Schematic overview of the procedure for RNA extraction from blood.

FIG. 2. Schematic overview of the procedure for RNA sequence library generation.

FIG. 3. Differential gene expression observed in immunodeficiency. (A) Differential gene expression analysis comparing 19,521 genes expressed in blood of PID patients and normal matched controls. Patients (n=20) diagnosed with PID who have antibody deficiency compared to healthy controls (n=20). Original source data collected by inventors. (B) Gene expression analysis identifies genes with down-regulated expression (x-axis expressed as log 2FoldChange) in blood of immunosuppressed patients after 3 months cyclosporin treatment. 50 significantly down regulated genes shown. (C-E) Volcano plots of differentially regulated gene sets in 4 immune deficiency conditions relative to normal control individuals. Black circles represent genes that are significantly regulated with a p value of <0.05 and a log 2 fold change of <−1 or >1 in expression. DE genes were identified using DESeq2 except where otherwise stated. Grey circles represent genes that do not meet this criteria and are deemed to be not differentially expressed. (C) An alternate representation of the data presented in (A)_above (D) Patients infected with HIV (n=32) compared to healthy controls. Data sourced from Verma et al, BMC Infect Dis. 2018; 18:220 (E) Cancer patients who have had their immune systems suppressed following treatment with cyclosporine, an immunosuppressant, for a period of 3 months. Data used for analysis was from measuring whole transcriptome >16,000 genes from Dorr et al. PLoS one, 2015; 10(5):e0125045. (F) Hematopoietic stem cell transplantation patients (n=10) compared to patients who have not undergone a transplant (n=13). Data sourced from Englert et al, Respir Res. 2019; 20:15.

FIG. 4. Application of a predictive SID model to evaluate SID susceptibility score on a set of individuals derived from their whole blood RNAseq expression profiles. The analysis demonstrates an example of variation in immune status of individuals where a high score may indicate weaker immune systems and therefore susceptibility to SID or sub-clinical SID under different therapy or treatment circumstances. (A) SID predictions scores on the x-axis from a study including diabetic patients at two time points. M2 labelled columns represents same individual, but following metformin drug treatment for 3 months. (calculated from supplementary data from Ustinova et al. PLoS one, 2020; 15(8):e0237400). (B) Application of a predictive SID model to evaluate SID susceptibility score on a set of individuals in an immunosuppression and bone marrow transplantation study. (Data used from Englert et al 2019 Whole blood RNA sequencing reveals a unique transcriptomic profile in patients with ARDS following hematopoietic stem cell transplantation). Patients 9, 11, 15, and 23 had very low IgA expression levels (determined by mRNA analysis) indicative of more severe immunosuppression status. (C) SID prediction score in 32 normal healthy controls. (D) SID prediction score for individual patients known to be infected in HIV. (E) SID prediction score for individual patients who have undergone hematopoietic stem cell transplantation (in black) and those who have not (in grey).

FIG. 5. Plot depicting how SID prediction scores can be measured over different time points in individual patients who underwent percutaneous osseointegrated lower limb implantation. Each shape represents a different patient at various time points including one week before and several time points after surgery.

FIG. 6. Chemotherapy that compromises the immune system significantly alters mouth microbial profiles of patients over time. (A) Analysis demonstrating example of significant differences in specific bacterial populations in the mouth microbiome of a group of patients before (dark bars) and after (light bars) undergoing combined radiotherapy and chemotherapy (cisplatin). Combined data from 6 patients. Data used for analysis was from supplementary data from Kumpitsch et al. Scientific reports, 2020; 10:16582. (B) Analysis demonstrating example of changes over time in specific bacterial populations in the mouth microbiome of a patient before (dark bars) and after (light bars) undergoing 45 days of combined radiotherapy and chemotherapy according to the example of the disclosure. (C) Analysis demonstrating significant changes in the mouth microbiome from patients diagnosed with PID compared to healthy controls.

FIG. 7. A Receiver Operating Characteristic (ROC) curve demonstrating the utility of the microbial SID prediction model. In this example, a microbial SID prediction model was developed using microbial diversity data derived from 20 patients clinically diagnosed with immunodeficiency and 20 healthy controls. Results from the application of the model to individuals using a leave out one cross validation method were used for constructing the ROC curve.

FIG. 8. Example of variant detection through whole blood RNAseq: Detection through RNAseq of a TNFRSF13B gene variant carried by an individual. RNAseq data obtained from whole blood of patient 32, cDNA sequence reads from the TNFRSF13B gene exon 3 mRNA sequence where a SNP substituted allele (in the position marked by the arrow) was observed different to the reference human genome sequence.

FIG. 9. A block diagram of a computer processing system configurable to perform various features of the present disclosure.

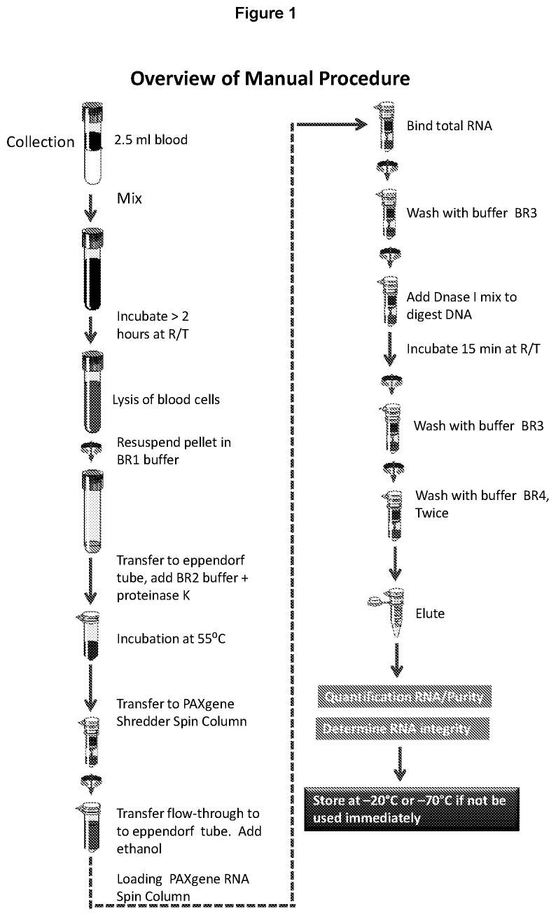

### DETAILED DESCRIPTION

A need exists to timely and accurately determine, detect, monitor or diagnose SID in a subject. The invention provides such a method that exploits RNAseq, and/or the metagenome, and a linear mixed model to predict, determine, detect, monitor or diagnose SID in a subject. This will have an impact on treatment decisions to improve survival and quality of life for patients, and a major public health impact by increasing rates and timeliness of diagnosis, thereby significantly reducing the cost of care for patients, and reduced demand on expensive pathology services.

“Secondary immunodeficiency disease” as used herein includes, but is not limited to: antibody deficiencies, combined immunodeficiencies, phagocytic cell deficiencies, immune dysregulation, or complement deficiencies that are caused by a variety of conditions including but not limited to: malnutrition; cancer; diabetes mellitus; chronic uremia; advanced age; lifestyle factors; genetic syndromes including but not limited to trisomy 21; anti-inflammatory, immunomodulatory and immunosuppressive drugs including but not limited to: corticosteroids, calcineurin inhibitors and cytotoxic drugs; surgery and trauma; environmental conditions including but not limited to: UV light, radiation and hypoxia; infectious diseases including but not limited to HIV infection; or any other cause of secondary immunodeficiency described herein.

In relation to cancer, SID may be caused by chemotherapy, radiotherapy, poor nutrition, cancers of the bone marrow or blood impact immune cell production (e.g. lymphoma, multiple myeloma and leukemia), removal of the spleen due to presence of a tumour, bone marrow or stem cell transplants or anaesthesia.

RNAseq provides at least the following three advantages over DNA analyses.

a) Mutation detection. An advantage for mutation detection in RNA over genomic DNA is that in RNA sequence only expressed genes are represented. The sequence does not include the vast majority of the genome sequence (98%) that is not expressed, thereby reducing the amount of total sequence generation required to identify mutations. This provides for a significant reduction in nucleic acid complexity (and increase in information density for increased throughput and efficiency) particularly if methods for depletion of abundantly expressed globin transcripts are applied before sequencing. The expressed gene sequences in blood are also enriched for expressed immune genes including their coding sequences. As a result less total sequence information needs to be obtained to determine mutation status. This enrichment in RNA of expressed and spliced genes from the genome results in less sequence required to be obtained and therefor lower sequencing costs. The increased relevance and focus of the sequence information obtained from RNA (with the reduced level of irrelevant sequence information) also improves the reliability and efficiency of bioinformatics processing.

b) RNA sequencing advantage for measuring SID gene transcript integrity. RNA sequencing has advantages over DNA sequencing in that it can be used to identify RNA structure variants e.g. splicing variants and misplaced intron expression. RNA sequencing can also identify abnormally low expression of a SID gene e.g. in blood as the result of; a transcript defect, destabilising mutation, or hard to identify regulatory region mutations preventing gene expression. The sequence represented in RNA can include coding RNAs and non-coding RNAs. Short read NGS technologies are well suited to this, however, long-read sequencing technologies, such as Pacific-Biosciences (PacBio) SMRT and Oxford Nanopore are suitable and have advantages for measuring transcript presence and integrity.

c) RNA sequencing advantage for measuring immune cell composition and activity. In addition to having advantages over DNA sequencing for mutation detection as a component of SID determination, detection or diagnosis, RNA sequencing provides functional information (not contained in a DNA sequence) as it includes a comprehensive measure of gene activities, in this case the activity of genes in immune cells in blood. A holistic analysis of gene expression levels can assist in identifying immune deficiency, since expression of many genes measured in blood or blood derived cells can identify deficient or abnormal gene expression concomitant with immune cell population and immune cell function changes. The inability of SID patients to fight infections are a direct result of such immune cell population and immune cell functional changes in blood, and these changes are expected to be evident in the RNA transcript profile.

Due to the involvement of different cell types, and the large number of immune genes subsequently or secondarily affected in deficiency, a whole transcriptome approach with an encompassing mixed model analysis such as Best Linear Unbiased Prediction (BLUP) or BayesR [5] is required, modified to use read counts or transformed read counts rather than SNP information (a straight forward modification). BLUP or BayesR is required to assess the full extent of distinguishing characteristics of SID patients. The immune function information provided in one step by RNA sequencing (if the information is captured with appropriate analysis) provides an advantage in cost, time, and resolution over combinations of immunological status assays usually required for SID determination, detection or diagnosis such as lymphocyte proliferation and cytotoxicity assays, flow cytometry, measurement of serum immunoglobulin levels, complete blood cell counts, neutrophil function tests, and complement assays.

RNAseq is used in disease research as it is useful for investigative purposes, but due to a number of difficulties RNAseq is not used in a clinical setting for determination, detection or diagnostic purposes or for the routine assessment of diseases [1]. Difficulties in being able to use whole transcriptome RNA expression information stem from the complexity of the information (data represented for 1000s of genes) and a lack of knowledge of the relevant components of the information (such as specific genes and pathways) to monitor expression levels of for determination, detection or diagnosis of diseases such as SID. Further to that, a lack of suitable statistical analysis approaches exists to identify and utilise putative mRNA biomarkers. Even when mRNA biomarkers in RNAseq data can be identified, a lack of standardisation for RNA sequence processing and defined statistical analysis limits potential clinical application.

More developed approaches exist for DNA sequencing, providing a more established paths and standards for mutation detection to complement clinical immune system information. RNA-sequencing for mutation detection in expressed gene sequences is useful, however, functional information that may be provided by the transcriptome sampling can be also be used. A BLUP or BayesR linear mixed model approach provides an analysis of the transcript abundance information in RNASeq data that permits it to be used directly as a diagnostic. Limitations of using RNA-sequencing alone w/o RNA expression BLUP or BayesR analysis are that mutations can be detected in expressed gene sequences, however functional information that may be provided by the RNA sequence profile/transcriptome data on the immune system is not fully captured and used.

A BLUP or BayesR model provides an approach that permits very many effects including small effects in cells and pathways (stemming from a deficiency in the immune system) to be incorporated into analysis and assessment for diagnosis. This approach may obviate the need for clinical immunological tests as it can capture a broad range of functional consequences at the RNA level. An approach taken for diagnosis discovery (not using BayesR or BLUP) would typically be to try and identify key genes as markers of function that could be used in place of clinical immunological tests. For example, cell composition changes in SID could be assessed by measuring transcripts for specific markers such as CD4, CD14, CD3, CD56, and CD19. Similarly other specific pathways or gene networks found to be affected in SID could also be used either as individual tests, combined tests, or by deriving individual gene set information from RNAseq data. BLUP and BayesR provide a solution as they can be applied directly utilising complete RNAseq information, and thereby incorporate large numbers of genes affected in the analysis, and can measure large numbers of small effects expected to occur as a result of SID mutations.

BLUP and BayesR approaches proposed by the inventors, have an advantage over other more targeted diagnostic marker approaches, as they use full information from the gene expression profiles (using all genes expressed in blood in the analysis) directly as the diagnostic signature, as opposed to using a single or more limited number of informative and/or known markers (if they were discovered and available to use for SID diagnosis application) as separate gene expression assays or deriving specific information from RNAseq data. In addition, the BLUP or BayesR approach is straightforward and efficient, requiring a single computational step without human intervention or requiring a combination of analysis methods. The transcriptomic BLUP or BayesR approach is also best suited to being able to identify a range of overlapping immune deficiency gene expression patterns reflecting disease from a diversity of causal mutations in different patients. A more limited set of diagnostic gene markers (if they were available) may not be able to identify a range of SID disease diversity. In addition, when trained on appropriate affected and non-affected patient reference profiles, the BLUP/BayesR approaches do not require specific knowledge of all aspects of the functional changes being measured for diagnosis in order to be implemented effectively, and therefore are able to capture informative consequences of mutations not yet understood to assist in the diagnosis.

The inventors have overcome difficulties by providing a sequencing and whole transcriptomic BLUP/BayesR methodology to determine, detect, monitor or diagnose SID. This obviates the need for the functional tests required for SID diagnosis by providing a method of contemporaneously assaying genomic information and immune cell function in one-step by molecular means. Avenues of investigation for improved functional tests mostly include expanding the cell types being examined using antibody markers and FACS, and examination of cells for defective function tested under activation conditions.

RNAseq is not contemplated as a diagnostic, but used as an investigative tool to identify genes and pathways associated with immune function. In this case, investigators would start by selecting certain genes from various analyses as candidates for immune function monitoring and diagnosis. For example, taking parallels from RNAseq application taken in other diseases, SID subject samples and the normal subject samples would potentially be compared by various means, and differentially expressed transcripts will be identified as different between the SID subjects' and normal subjects' samples. Gene ontology enrichment analysis would be performed using tools such as the DAVID website (https://david.ncifcrf.gov/). The differential gene expression profile could also be subjected to gene set enrichment analysis using gene set enrichment analysis (GSEA) with MSigDB public immunological gene signatures. Investigators are likely to perform RNAseq for investigative purposes and search in RNAseq data for known genes and pathways, or known cell markers, typically conducting RNAseq on subsets of blood cells. A BLUP approach on RNAseq from whole blood is able to incorporate information from known, and unknown gene networks that are not well understood, where direct and indirect effects can be captured has not been envisaged as a direct diagnostic and not as a surrogate for a range of cell-based assays. Nowhere is it suggested that whole blood transcriptomic BLUP be used directly as a diagnostic to replace cell and immune function assays including for SID.

BLUP has been used to classify samples into subsets to aid in investigative studies, and enhance genetic diagnosis (SNP variance) for multi-genic diseases. In some cases, BLUP can be used to combine diverse types of clinical information to provide more accurate prognosis. Application of BLUP for disease classification has been applied in neuroblastoma [6].

Other clinical information can be used in combination with information from the RNA-based methods described above to assist in diagnosis, including microbial colonisation information. Recording and managing infections including in some cases microbial diagnostic approaches for organisms that are pathogenic are an important component of SID diagnosis.

Metagenomic sequencing extends the analysis of microbial compositions beyond pathogens with information that includes a comprehensive measure of microbial community activities. A holistic analysis of microbial interface management will be able to assist in identifying immune deficiency, since the presence of many organisms in mucosa or hair follicles can identify deficient, or abnormal community structures, or combinations of specific organisms concomitant with immune cell population and immune cell function changes.

As used herein, “RNAseq” or “transcriptome” refers to genes expressed and then sequenced, the sequence reads of which align to exon sequences in the genome or a reference transcriptome database. A “transcriptome profile” is the vector of counts of the sequence reads, and accordingly, is the overall, characterizing composition of genes expressed in a sample.

A transcriptomic relationship matrix may be generated from transcriptome profiles as set out in the examples, and may be generated as part of the method of the invention or may be pre-existing.

In one embodiment of the present invention, the linear mixed model is BLUP or BayesR. As used herein, a “linear mixed model”, also called a “multilevel model” or a “hierarchical model”, refers to a class of regression models that takes into account both the variation that is explained by the independent variables of interest and the variation that is not explained by the independent variable of interest, or random effects. Examples of linear mixed models include, but are not limited to, BayesR and best linear unbiased prediction (BLUP). The person skilled in the art will be aware of other appropriate linear mixed models.

In one embodiment, the SID prediction equation is any one described herein, including the Example.

The predictive scores (either relative or absolute) generated may be used to classify subjects into high (e.g. a score where a higher value is high risk) or low (e.g. a score where a lower value is low risk) risk of having SID. For example, when using an absolute predictive score, a score of >0.2 provides a diagnostic assay for detection of SID with a certain level of sensitivity and specificity. A score of >0.4 provides a diagnostic assay for detection of SID with a decreased sensitivity and increased specificity. A score of >0.6 provides a diagnostic assay for detection of SID with a further decrease in sensitivity and a further increase in specificity. In contrast, for example, when using a relative predictive score (whereby a relative predictive score for each patient when matched with a control group is determined by subtracting the healthy control score from the patient score), a score of >0, >0.1 and >0.2 provides a diagnostic assay for detection of SID.

In another embodiment the predictive score may be used to monitor the status of SID in a subject. For example, changes to SID status are relative to a reference score for each patient (whereby the reference score for each patient is compared to a repeat score at different time and the change in SID status is determined by subtracting the repeat score from the reference score). A score of >0.1 indicates a higher risk category for SID for the patient being monitored. A score of <0 indicates a low risk of SID or an improvement in immune function.

In one embodiment of the present invention, the reference set further comprises an RNA sequence mutation profile. In a further embodiment of the present invention, the reference set further comprises an RNA sequence mutation profile and the linear mixed model is used to fit the transcriptome profile and a RNA sequence mutation profile of the subject to the SID prediction equation.

In one embodiment of the present invention, the reference set further comprises a DNA sequence mutation profile. In a further embodiment of the present invention, the reference set further comprises a DNA sequence mutation profile and the linear mixed model is used to fit the transcriptome profile and a DNA sequence mutation profile of the subject to the SID prediction equation.

In one embodiment of the present invention, the reference set further comprises a metagenome profile. In a further embodiment of the present invention, the reference set further comprises a metagenome profile and the linear mixed model is used to fit the transcriptome profile and a metagenome profile of the subject to the SID prediction equation.

The term “Metagenome” as used herein refers to the total DNA recovered from a sample, including the DNA from microbial inhabitants or the “microbiome” of the sample. “Metagenome profile” as used herein refers to the overall, characterizing composition of microbial DNA in a sample. “Microbiome” as used herein refers to all of the microbes in a sample.

In one embodiment of the method of the present invention, the metagenome profile is obtained from a mouth swab, nose swab, throat swab, saliva, faeces, skin, or a hair follicle. That is, the metagenome profile is obtained from a sample that comprises the microbiome from a mouth swab, nose swab, throat swab, saliva, faecal sample, skin sample or hair follicle sample.

In another embodiment of the method of the present invention, the metagenome profile sample is obtained by a consumer comprising the microbiome from a mouth swab, nose swab, throat swab or saliva sample.

In a further embodiment of the method of the present invention, consumers are provided with a score based on the metagenome profile of the mouth microbiome, which is monitored during lifestyle changes such as pregnancy, drugs and new nutrition.

The term “Gene sequence mutation” as used herein encompasses both an RNA sequence mutation and a DNA sequence mutation, and refers to a change from the wild-type or a reference sequence of one or more nucleic acid molecules. “Mutations” include without limitation, base pair substitutions, additions and deletions of at least one nucleotide from a nucleic acid molecule of known sequence. A mutated nucleic acid can be expressed from or found on one allele (heterozygous) or both alleles (homozygous) of a gene, and may be somatic or germ line. Accordingly, a “gene sequence mutation profile” is the overall, characterizing composition of gene sequence mutations in a sample.

A gene sequence mutation also encompasses:
a) where the RNA sequence of a gene is shown to have such a known mutation associated with a SID;
b) where a new mutation (e.g. a missense mutation resulting in an amino acid change or nonsense mutation resulting a frameshift) affecting a predicted structure or function of a protein is detected in a known SID-associated gene from the RNA sequence;
c) where a dominant mutation is detected in one allele from the RNA sequences;
d) where two different mutations occur in the same gene, but on two different alleles;
e) where a known mutation in RNA is inferred or imputed from linkage to a co-occurring haplotype marker in the RNA expressed from the same gene, or nearby gene on the chromosome;
f) where expression of a SID-associated gene sequence normally expressed in blood is not detected in blood RNA (indicating a serious regulatory defect or destabilising mutation);
g) where the exon structure of the mutated SID-associated gene determined by RNAseq is defective (indicating a splicing defect);
h) where one or more (1-3) additional SID-associated gene mutations are detected in the same patient from the RNA/cDNA sequence; and
i) where the sequence of several other genes, or imputed sequence of other genes, detected in the RNA profile contributes to SID severity.

Expressed differently, in one embodiment of the method of the present invention, the mutation profile comprises:
a) a RNA sequence of a gene comprising a mutation associated with a SID;
b) a new mutation, optionally a frameshift mutation, that affects structure or function of a protein encoded by a known gene mutation of which associates with a SID;
c) a dominant mutation in one allele associated with a SID;
d) two different mutations in the same gene, but on two different alleles associated with a SID;
e) a known mutation in RNA that is inferred or imputed by linkage to a co-occurring marker for a mutation associated with a SID;
f) absence of expression of a gene normally expressed in non-SID subjects indicating a regulatory defect or destabilising mutation;
g) a defective exon structure indicating a splicing defect;
h) one or more, optionally one to three, additional mutations associated with a SID; or
i) a sequence of more than one other gene, or an imputed sequence of more than one other gene, that associates with SID severity.

As used herein, “reference set” or “training set” refers to a single or group of transcriptome profiles, gene sequence mutation profiles, or metagenome profiles obtained from subjects with and/or without SID, i.e. “reference subject(s)”, used to generate a transcriptomic relationship matrix, subsequently used to predict or monitor SID.

The term “marker” or “biomarker” as used herein refers to a biochemical, genetic (either DNA or RNA), or molecular characteristic that is a surrogate for and therefore indicative/predictive of a second characteristic, for example a genotype, phenotype, pathological state, disease or condition.

In one embodiment of the present invention, the transcriptome profile or sequence mutation profile is obtained from sputum, blood, amniotic fluid, plasma, semen, bone marrow, tissue, urine, peritoneal fluid, or pleural fluid, optionally obtained by fine needle biopsy. In a further embodiment, the blood comprises peripheral blood mononuclear cells.

A “subject” as used herein may be human or a non-human animal, for example a domestic, a zoo, or a companion animal. In one embodiment, the subject is a mammal. The mammal may be an ungulate and/or may be equine, bovine, ovine, canine, or feline, for example. In one embodiment, the subject is a primate. In one embodiment, the subject is human. Accordingly, the present invention has human medical applications, and also veterinary and animal husbandry applications, including treatment of domestic animals such as horses, cattle and sheep, and companion animals such as dogs and cats.

Throughout the description and claims of this specification, the word “comprise” and variations of the word, such as “comprising” and “comprises”, means “including but not limited to”, and is not intended to exclude other additives, components integers or steps.

As used herein, “determining whether a subject has or is susceptible to developing a SID” refers to detecting or diagnosing a SID in a subject, or predicting or prognosing, that a subject is likely to develop a SID. The invention also encompasses detecting a SID in a subject or detecting susceptibility to a SID in a subject. In some embodiments the subject for whom the determination of SID or susceptibility to SID are potential workers in high risk infectious environments including but not limited to: defence personnel and professionals working with infectious diseases. Determining susceptibility to a SID includes determining future susceptibility to SID.

The term “monitor the status of SID in a subject” as used herein refers to the longitudinal monitoring of SID in a subject who has or is susceptible to developing a SID. The invention encompasses detecting change in SID status, determined by changes in the SID predictive score of a subject, which may indicate the development of SID or conversely improved immunity in a patient. Certain aspects of the invention involve determining transcriptome profiles, or other profile described herein, in samples obtained over a period of time. Repeat, further or subsequent samples are obtained at a later time than an initial sample at the start of monitoring, wherein the repeat, further or subsequent sample may be obtained following an exposure event (e.g. exposure to an agent as described herein) or risk factor that could give rise to SID or in the routine monitoring of SID. In other words, the invention encompasses determining, detecting, monitoring or diagnosing a SID in a subject and/or determining, detecting, monitoring or diagnosing susceptibility to a SID in a subject.

As used herein, the terms “subclinical SID” refers to individuals who do not have any clinically detectable symptoms of SID but where their immune system health may be affected by lifestyle factors such as exercise, sickness, infection, stress, toxins, recreational drugs, medicines, fitness, weight, diet, pregnancy, supplements, programs, treatments, environment changes, probiotics, anti-inflammatoires, antibiotics, glucocorticoids, gum disease, or surgery. A measure for immune health (subclinical or predisposition to SID) could be whole transcriptome predictions of serum or mucosal antibody levels such as levels of IgG1 or IgA1/IgA2 respectively.

The term “biological sample” as used herein refers to a sample which may be tested for a particular “gene expression profile”, “gene sequence mutation profile”, “transcriptome profile” or “sequence mutation profile” (where the sequence mutation profile may be mutation in RNA and/or DNA). A sample may be obtained from an organism (e.g. a human patient) or from components (e.g. cells) of an organism. The sample may be of any relevant biological tissue or fluid which comprises RNA and/or DNA. The sample may be a “clinical sample” which is a sample derived from a patient. Such samples include, but are not limited to, sputum, blood, blood cells (e.g. white cells), amniotic fluid, plasma, semen, bone marrow, and tissue or fine needle biopsy samples, urine, peritoneal fluid, and pleural fluid, or cells therefrom. Biological samples may also include sections of tissues such as frozen sections taken for histological purposes. A biological sample may also be referred to as a “patient sample”. In one embodiment, the method of the invention is not practiced on a human or animal body, for example, the test profile may be determined by analysing previously obtained biological sample.

The term “gene” as used herein refers to a nucleic acid sequence that comprises coding sequences necessary for producing a polypeptide or precursor. Control sequences that direct and/or control expression of the coding sequences may also be encompassed by the term “gene” in some instances. The polypeptide or precursor may be encoded by a full-length coding sequence or by a portion of the coding sequence. A gene may contain one or more modifications in either the coding or the untranslated regions that could affect the biological activity or the chemical structure of the polypeptide or precursor, the rate of expression, or the manner of expression control. Such modifications include, but are not limited to, mutations, insertions, deletions, and substitutions of one or more nucleotides, including single nucleotide polymorphisms that occur naturally in the population. The gene may constitute an uninterrupted coding sequence or it may include one or more subsequences.

The term “gene expression level” or “expression level” as used herein refers to the amount of a “gene expression product” or “gene product” in a sample. “Gene expression profile” or “gene expression signature” as used herein refers to a group of “gene expression products” or “gene products” produced by a particular cell or tissue type wherein expression of the genes taken together, or the differential expression of such genes, is indicative and/or predictive of a pathological state, disease or condition, such as an immune disorder. A “gene expression profile” can be either qualitative (e.g. presence or absence) or quantitative (e.g. levels or mRNA copy numbers). Thus, a “gene expression profile” can also be used to determine the numbers of specific cell types in a heterogeneous sample of cells, such as the number of T cells in a blood sample, based on the amount of cell-type specific “gene expression products” or “gene products”.

The term “gene expression product” or “gene product” as used herein refers to the RNA transcription products (RNA transcript) of a gene, including mRNA, and the polypeptide translation product of such RNA transcripts. A “gene expression product” or “gene product” can be, for example, a polynucleotide gene expression product (e.g. an un-spliced RNA, an mRNA, a splice variant mRNA, a microRNA, a fragmented RNA) or a protein expression product (e.g. a mature polypeptide, a splice variant polypeptide).

The term “immune cell” as used herein refers to cells, such as lymphocytes, including natural killer cells, T cells, B cells, macrophages and monocytes, dendritic cells or any other cell which is capable of producing an “immune effector molecule” in response to direct or indirect antigen stimulation. The term “immune effector molecules” are molecules which are produced in response to cell activation or stimulation by an antigen, including, but not limited to, cytokines such as interferons (IFN), interleukins (IL), such as IL-2, IL-4, IL-10 or IL-12, tumor necrosis factor alpha (TNF-α), colony stimulating factors (CSF), such as granulocyte (G)-CSF or granulocyte macrophage (GM)-CSF, complement and components in the complement pathway.

The term “immune disorder” as used herein refers to a pathological state, disease or condition characterized by a dysfunction in the immune system. “Immune disorders” include, but are not limited to, autoimmune disorders, such as scleroderma, allergies, such as allergic rhinitis, and immunodeficiencies, such as secondary immunodeficiency disease.

The term “normal immune system” as used herein refers to an immune system that has a normal composition of immune cells and wherein said immune cells are not dysfunctional. A “normal” or “healthy” subject as used herein refers to a subject with a “normal immune system”.

The term “nucleic acid” as used herein refers to DNA molecules (e.g. cDNA or genomic DNA), RNA molecules (e.g. mRNA), DNA-RNA hybrids, and analogs of the DNA or RNA generated using nucleotide analogs. The nucleic acid molecule can be a nucleotide, oligonucleotide, double-stranded DNA, single-stranded DNA, multi-stranded DNA, complementary DNA, genomic DNA, non-coding DNA, messenger RNA (mRNA), microRNA (miRNA), small nucleolar RNA (snoRNA), ribosomal RNA (rRNA), transfer RNA (IRNA), small interfering RNA (siRNA), heterogeneous nuclear RNAs (hnRNA), or small hairpin RNA (shRNA).

A method of the present invention may comprise the further step of treating a SID in a subject determined to have or be susceptible to a SID.

Accordingly, also disclosed is treatment of a SID in a subject determined to have or be susceptible to a SID by a method of the invention.

Accordingly, disclosed herein is a method of treating a SID in a subject, the method comprising:
administering to the subject an antibiotic, an immunoglobulin, an interferon, a growth factor, gene therapy, or enzyme replacement therapy; or
transplanting a hematopoietic stem cell into the subject,
wherein the subject is determined to have or be susceptible to developing SID by the method of the present invention.

Also disclosed is use of an antibiotic, an immunoglobulin, an interferon, a growth factor, an enzyme, a gene, or a hematopoietic stem cell in the manufacture of a medicament for treating a SID in a subject, wherein the subject is determined to have or be susceptible to developing SID by the method of the present invention.

Also disclosed is an antibiotic, an immunoglobulin, an interferon, a growth factor, an enzyme, a gene, or a hematopoietic stem cell for use in a method of treating SID in a subject, wherein the subject is determined to have or be susceptible to developing SID by the method of the present invention.

For SID determination, detection or diagnosis, RNAseq provides three main advantages over DNA sequencing for mutation detection and immune function assessment: (a) mutations are detected in expressed genes only; (b) SID gene transcript integrity; (c) immune cell composition and activity.

In one embodiment, the SID to be treated is selected from: antibody deficiencies, combined immunodeficiencies, phagocytic cell deficiencies, immune dysregulation, or complement deficiencies that is caused by a variety of conditions including but not limited to: malnutrition; diabetes mellitus; chronic uremia; advanced age; genetic syndromes including but not limited to trisomy 21; anti-inflammatory, immunomodulatory and immunosuppressive drugs including but not limited to: corticosteroids, calcineurin inhibitors and cytotoxic drugs; surgery and trauma; environmental conditions not limited to: UV light, radiation and hypoxia; infectious diseases including but not limited to HIV infection; or any other cause of secondary immunodeficiency described herein.

Effective treatments of SIDs include managing infection, boosting the immune system, hematopoietic stem cell transplantation, gene therapy, and enzyme replacement therapy.

Managing infections includes:
treating infections with antibiotics, usually rapidly and aggressively-infections that do not respond may require hospitalization and intravenous (IV) antibiotics.
preventing infections, for example with long-term antibiotic treatment to prevent respiratory infections and associated permanent damage to the lungs and ears, and avoidance of vaccinating children with SID using vaccines containing live viruses, such as oral polio and measles-mumps-rubella.
treating symptoms using pharmaceutical substances such as ibuprofen for pain and fever, decongestants for sinus congestion, expectorants to thin mucus in the airways, or using postural drainage in which gravity and light blows are applied to the chest to clear the lungs.

Boosting the immune system includes:
immunoglobulin therapy, usually intravenously every few weeks or subcutaneously once or twice a week.
gamma interferon therapy to combat viruses and stimulate immune cells, usually intramuscularly three times a week, most often to treat chronic granulomatous disease.
growth factor therapy to increase the levels of white blood cells.

Stem cell transplantation may be a treatment for several forms of life-threatening SID.

It will be appreciated by the person skilled in the art that the exact manner of administering to a subject a therapeutically effective amount of an antibiotic, an immunoglobulin, an interferon, a growth factor, hematopoietic stem cells, a gene for gene therapy, or an enzyme for enzyme replacement therapy will be at the discretion of the medical practitioner with reference to the SID to be treated or prevented. The mode of administration, including dosage, combination with other agents, timing and frequency of administration, and the like, may be affected by the subject's likely responsiveness to treatment, as well as the subject's condition and history.

The antibiotic, immunoglobulin, interferon, growth factor, hematopoietic stem cells, gene for gene therapy, or enzyme for enzyme replacement therapy will be formulated, dosed, and administered in a fashion consistent with good medical practice. Factors for consideration in this context include the particular SID being treated or prevented, the particular subject being treated, the clinical status of the subject, the site of administration, the method of administration, the scheduling of administration, possible side-effects and other factors known to medical practitioners. The therapeutically effective amount of antibiotic, immunoglobulin, interferon, growth factor, hematopoietic stem cells, gene for gene therapy, or enzyme for enzyme replacement therapy to be administered will be governed by such considerations.

The antibiotic, immunoglobulin, interferon, growth factor, hematopoietic stem cells, gene for gene therapy, or enzyme for enzyme replacement therapy may be administered systemically or peripherally, for example by routes including intravenous (IV), intra-arterial, intramuscular (IM), intraperitoneal, intracerobrospinal, subcutaneous (SC), intra-articular, intrasynovial, intrathecal, intracoronary, transendocardial, surgical implantation, topical and inhalation (e.g. intrapulmonary).

The term “therapeutically effective amount” refers to an amount of antibiotic, immunoglobulin, interferon, growth factor, hematopoietic stem cells, gene for gene therapy, or enzyme for enzyme replacement therapy effective to treat a SID in a subject.

The terms “treat”, “treating” or “treatment” refer to both therapeutic treatment and prophylactic or preventative measures, wherein the aim is to prevent or ameliorate a SID in a subject or slow down (lessen) progression of a SID in a subject. Subjects in need of treatment include those already with the SID as well as those in which the SID is to be prevented.

The terms “preventing”, “prevention”, “preventative” or “prophylactic” refers to keeping from occurring, or to hinder, defend from, or protect from the occurrence of a SID, including an abnormality or symptom. A subject in need of prevention may be prone to develop the SID.

The term “ameliorate” or “amelioration” refers to a decrease, reduction or elimination of a SID, including an abnormality or symptom. A subject in need of amelioration may already have the SID, or may be prone to develop the SID, or may be in whom the SID is to be prevented.

In another embodiment a method of the present invention may comprise the further step of treatment of a SID in a subject where an agent has caused the SID.

Accordingly, also disclosed is treatment of a SID in a subject for whom the longitudinal monitoring of SID is to be made and the subject has or develops a SID.

Accordingly, disclosed herein is a method of treating a SID in a subject where an agent has caused the SID, the method comprising:
reducing the dose of the agent

wherein the subject is determined to have developed a SID by a method of the present invention.

The term “agent” refers to any compound that reduces the function of the immune system, preferably one that is used in the treatment of a disease or condition. For example, some pharmaceutical substances used in the treatment or prevention of a disease may contribute to the development of a SID such as with the use of cytotoxic agents and immunosuppressants.

In one embodiment the SID is caused by an agent used in the treatment or prevention of cancer or graft versus host disease.

In a preferred embodiment the SID is caused by an agent which is a chemotherapy or corticosteroid.

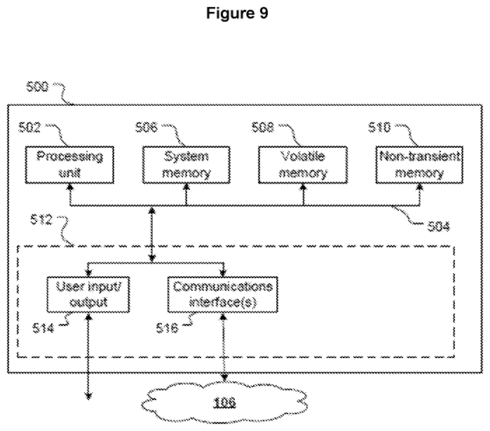

FIG. 9 provides a block diagram of a computer processing system 500 configurable to implement embodiments and/or features described herein. System 500 is a general purpose computer processing system. It will be appreciated that FIG. 9 does not illustrate all functional or physical components of a computer processing system. For example, no power supply or power supply interface has been depicted, however system 500 will either carry a power supply or be configured for connection to a power supply (or both). It will also be appreciated that the particular type of computer processing system will determine the appropriate hardware and architecture, and alternative computer processing systems suitable for implementing features of the present disclosure may have additional, alternative, or fewer components than those depicted.

Computer processing system 500 includes at least one processing unit 502—for example a general or central processing unit, a graphics processing unit, or an alternative computational device). Computer processing system 500 may include a plurality of computer processing units. In some instances, where a computer processing system 500 is described as performing an operation or function all processing required to perform that operation or function will be performed by processing unit 502. In other instances, processing required to perform that operation or function may also be performed by remote processing devices accessible to and useable by (either in a shared or dedicated manner) system 500.

Through a communications bus 504, processing unit 502 is in data communication with a one or more computer readable storage devices which store instructions and/or data for controlling operation of the processing system 500. In this example system 500 includes a system memory 506 (e.g. a BIOS), volatile memory 508 (e.g. random access memory such as one or more DRAM modules), and non-volatile (or non-transitory) memory 510 (e.g. one or more hard disk or solid state drives). Such memory devices may also be referred to as computer readable storage media.

System 500 also includes one or more interfaces, indicated generally by 512, via which system 500 interfaces with various devices and/or networks. Generally speaking, other devices may be integral with system 500, or may be separate. Where a device is separate from system 500, connection between the device and system 500 may be via wired or wireless hardware and communication protocols, and may be a direct or an indirect (e.g. networked) connection.

Wired connection with other devices/networks may be by any appropriate standard or proprietary hardware and connectivity protocols, for example Universal Serial Bus (USB), eSATA, Thunderbolt, Ethernet, HDMI, and/or any other wired connection hardware/connectivity protocol.

Wireless connection with other devices/networks may similarly be by any appropriate standard or proprietary hardware and communications protocols, for example infrared, BlueTooth, WiFi; near field communications (NFC); Global System for Mobile Communications (GSM), Enhanced Data GSM Environment (EDGE), long term evolution (LTE), code division multiple access (CDMA—and/or variants thereof), and/or any other wireless hardware/connectivity protocol.

Generally speaking, and depending on the particular system in question, devices to which system 500 connects—whether by wired or wireless means—include one or more input/output devices (indicated generally by input/output device interface 514). Input devices are used to input data into system 100 for processing by the processing unit 502. Output devices allow data to be output by system 500. Example input/output devices are described below, however it will be appreciated that not all computer processing systems will include all mentioned devices, and that additional and alternative devices to those mentioned may well be used.

For example, system 500 may include or connect to one or more input devices by which information/data is input into (received by) system 500. Such input devices may include keyboards, mice, trackpads (and/or other touch/contact sensing devices, including touch screen displays), microphones, accelerometers, proximity sensors, GPS devices, touch sensors, and/or other input devices. System 500 may also include or connect to one or more output devices controlled by system 500 to output information. Such output devices may include devices such as displays (e.g. cathode ray tube displays, liquid crystal displays, light emitting diode displays, plasma displays, touch screen displays), speakers, vibration modules, light emitting diodes/other lights, and other output devices. System 500 may also include or connect to devices which may act as both input and output devices, for example memory devices/computer readable media (e.g. hard drives, solid state drives, disk drives, compact flash cards, SD cards, and other memory/computer readable media devices) which system 500 can read data from and/or write data to, and touch screen displays which can both display (output) data and receive touch signals (input).

System 500 also includes one or more communications interfaces 516 for communication with a network, such as the Internet in environment 100. Via a communications interface 516 system 500 can communicate data to and receive data from networked devices, which may themselves be other computer processing systems.

System 500 stores or has access to computer applications (also referred to as software or programs)—i.e. computer readable instructions and data which, when executed by the processing unit 502, configure system 500 to receive, process, and output data. Instructions and data can be stored on non-transitory computer readable medium accessible to system 500. For example, instructions and data may be stored on non-transitory memory 510. Instructions and data may be transmitted to/received by system 500 via a data signal in a transmission channel enabled (for example) by a wired or wireless network connection over interface such as 512.

Applications accessible to system 500 will typically include an operating system application such as Microsoft Windows®, Apple OSX, Apple IOS, Android, Unix, or Linux.

In some cases part or all of a given computer-implemented method will be performed by system 500 itself, while in other cases processing may be performed by other devices in data communication with system 500.

The transcriptome differences form a genetic signature for the disease can be identified using a learning software algorithm and proprietary reference database.

The genomic algorithm generates a predictive score which can be used to identify patients with disease. A component of the software is comprised of a bioinformatic pipeline that searches for specific gene mutations which have previously established to be associated with the risk of developing a SID.

The present invention includes methods described herein and a program that can utilise a set of existing bioinformatics tools (including R for the transcriptomic relationship matrix and BLUP prediction, and GATK) for mutation detection associated with the risk of developing a SID.

The invention will now be described with reference to the following, non-limiting examples.

### Example

Un-Blinded, Ex Vivo, Study Using Samples from Subjects with Confirmed Primary Immunodeficiency Disease and Normal Subjects.

The inventors believe that the example predictive models used to determine, detect and diagnosis primary immunodeficiency are directly applicable to secondary immunodeficiency for the uses described within this specification.

The predictive models analyse a large number of genes expressed to identify a signature of immunodeficiency. The underlying genetic defects and immune pathways causing PID leads to reductions in gene expression due to missing immune cells (such as B cells) or defective pathways (where gene expression is downregulated) and compensatory pathways (where gene expression is upregulated). Similarly, the causes of SID are variable but impact specific components of the immune system resulting in reduced cell numbers or reduced function. For example, in PID and SID a lower level of antibodies and antibody transcripts in many cases can be expected. For example, HIV infection directly impacts on the functioning of CD4 cells and then downstream functions of the immune system controlled by CD4 T cells such as B cells.

### Study Outline

An un-blinded, multi-centred, ex vivo, study using biological samples collected from 20 subjects with confirmed primary immunodeficiency disease (PID) and 20 normal subjects.

The study demonstrated that PID may be diagnosed using:
(i) gene expression data, i.e. RNAseq or the transcriptome, obtained by RNA sequencing;
(ii) gene expression data, i.e. RNAseq or the transcriptome, combined with gene sequence data;
(iii) gene expression data, i.e. RNAseq or the transcriptome, combined with microbial metagenome data, obtained by targeted or untargeted massively parallel sequencing and using a linear mixed model prediction approach can alone be used to diagnose PID; or
(iv) microbial metagenome data,

obtained by targeted or untargeted massively parallel sequencing and using a linear mixed model prediction.

### Exclusion Criteria

Subjects who have previously undergone a haematopoietic stem cell transplant were excluded from the study.

### Sample Collection

Blood cells from peripheral venous whole blood were collected for RNA extraction. Microbial samples were collected from mouth buccal swab, nose swab, throat swab, saliva, faecal sample, skin sample or hair follicle sample for DNA extraction.

### Transcriptome Profile Determination

RNA sequencing was performed to identify gene sequence mutations indicative of PID, and to determine the gene expression profile of PID subjects for comparison to normal subjects.

### i) Sample Collection and RNA Extraction

Blood cells from peripheral venous whole blood were prepared using PAXgene™ blood RNA tubes (PAXgene Blood RNA Kit (50)-Cat No./ID: 762164) according to the manufacturer's instructions. The reagent composition of PAXgene Blood RNA Tubes protects RNA molecules from degradation and can stabilise cellular RNA of human whole blood up to 3 days at 18-25° C. or up to 5 days at 2-8° C. or at 8 years at −20° C./−70° C.

2.5 ml of drawn blood was collected into PAXgene blood RNA tubes and incubated for at least 2 hours at room temperature to ensure complete lysis of blood cells. If the PAXgene Blood RNA Tube was stored at 2-8° C., −20° C. or −70° C. after blood collection, the sample is first equilibrated to room temperature, and then stored at room temperature for 2 hours before starting the procedure. After preparing buffers, the following steps were taken:

(i) Centrifuge the PAXgene Blood RNA Tube for 10 minutes at 3000-5000×g using a swing-out rotor and remove the supernatant.

(ii) Add 4 ml RNase-free water to the tube and close it using a fresh BD Hemogard Closure supplied with the kit.

(iii) Vortex until the pellet is visibly dissolved. Centrifuge for 10 minutes at 3000-5000×g using a swing-out rotor and remove the supernatant completely.

(iv) Add 350 μl Buffer BR1 and vortex until the pellet is visibly dissolved.

(v) Remove the sample into a 1.5 ml eppendorf tube. Successively add 300 μl buffer BR2 and 40 μl proteinase K. Mix by vortexing for seconds.

(vi) Incubate for 10 minutes at 55° C. using a shaker-incubator at 400-1400 rpm.

(vii) Pipet the lysate directly into a PAXgene Shredder spin column (lilac) placed in a 2 ml collecting tube and centrifuge for 3 minutes at maximum speed (but not to exceed 20 000×g, which may damage the columns).

(viii) Carefully transfer the entire supernatant of the flow-through fraction to a fresh 1.5 ml tube without disturbing the pellet in the processing tube.

(ix) Add 350 μl ethanol (96-100%, purity grade p.a.). Mix by vortexing and centrifuge briefly to remove drops from the inside of the tube lid.

(x) Pipet 700 μl into the PAXgene RNA spin column (red) placed in a 2 ml processing tube and centrifuge for 1 minute at 16000×g (8000-20,000×g). Discard the flow-through.

(xi) Pipette the remaining sample into the PAXgene RNA spin column and centrifuge for 1 minute at 16000×g (8000-20,000×g). Discard flow-through.

(xii) Wash the column with 350 μl Buffer BR3 into. Centrifuge for 1 minute at 16 000×g (8000-20 000×g).

(xiii) Add 80 μl DNase I mix (80 μl) directly onto the centre of PAXgene RNA spin column membrane and incubate at room temperature (20-30° C.) for 15 minutes.

(xiv) Pipet 350 μl Buffer BR3 into the PAXgene RNA spin column and centrifuge for 1 minute at 16 000×g (8000-20 000×g). Discard flow-through.

(xv) Wash the column with 500 μl BR4 and centrifuge for 1 minute at 16 000×g (8000-20 000×g). Discard the flow-through and centrifuge for another 1 minute at 16 000×g (8000-20 000×g).

(xvi) Add another 500 μl Buffer BR4 to the column and centrifuge for 3 minutes at 16 000×g (8000-20 000×g). Discard the processing tube containing the flow-through, and place the PAXgene RNA spin column in a new 2 ml processing tube. Centrifuge for 2 minute at 16 000×g (8000-20 000×g). Transfer the column in a 1.5 ml tube.

(xvii) Add 40 μl Buffer BR5 directly onto the column membrane. To elute RNA by centrifuging for 2 min at 16 000×g (8000-20 000×g). (Note: It is important to the centre of PAXgene RNA spin column for wetting the entire membrane with Buffer BR5 in order to achieve maximum elution efficiency.)

(xviii) Quantitate RNA/Purity, e.g. using NanaDrop 1000/2000 or Qubit instruments and using RNA specific binding fluorescent dye such as Quant-iT™ RNA.

(xix) Determine RNA integrity, e.g. using a BioAnalyser 2100 or TapeStation 2200 instrument (Agilent Technologies)

(xx) If the RNA samples will not be used immediately, store at −20° C. or −70° C.

### ii) RNA Sequencing

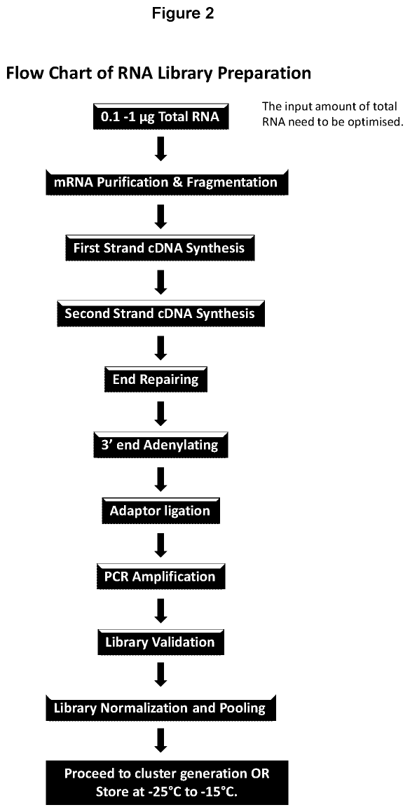

RNAseq libraries were prepared using the TruSeq RNA sample preparation kit (Illumina) according to the manufacturer's protocol outlined in FIG. 2.

Preparation of the whole transcriptome sequencing library was conducted using Illumina's “TruSeq Stranded Total RNA Library Prep Kit with Ribo-Zero Globin Set” according to manufacturer's instructions.

Multiplexes of libraries each with one of 12 indexed adaptors, were pooled. Each pool was sequenced on one flowcell lane on the HiSeq2000 sequencer (Illumina) in a 101 cycle paired end run.

iii) Gene Expression Profile Generation and Sequence Analysis

100 base long paired end-reads generated by the HiSeq2000 sequencer (Illumina) were called with CASAVA v1.8 and output in fastq format. Sequence quality was assessed using trimmomatic (v0.39) and scripts were used to trim and filter poor quality bases and sequence reads. Bases with quality score less than 20 were trimmed from 3′ end of reads. Reads with mean quality score less than 20, or greater than 3 N, or final length less than 35 bases were discarded. Only paired reads were retained for alignment.

After RNA-sequencing use of the Trimmomatic software [7], raw read sequences were trimmed for minimum quality at 3′ end (phred score of at least 30), cleaned of adapter traces and filtered for a final minimum length of 32 bp. Alignment to the Ensembl GRCh38.84 was performed using hisat2 (v2.1) or alternatively UCSC hg38 reference genome (Illumina iGenomes) sequence was performed using TopHat2 [8] (hg19 reference genome could be used). The merge of lanes and mark of duplicates was performed with gatk (v4.1.2.0. QC and quantification with RNAseQC (v2.3.4) GENCODE v24 annotation, modified according to GTEx collapse gene model. Differential gene expression was conducted with edgeR (v3.26.4.) after gene expression is quantified by counting the number of uniquely mapped reads [9].

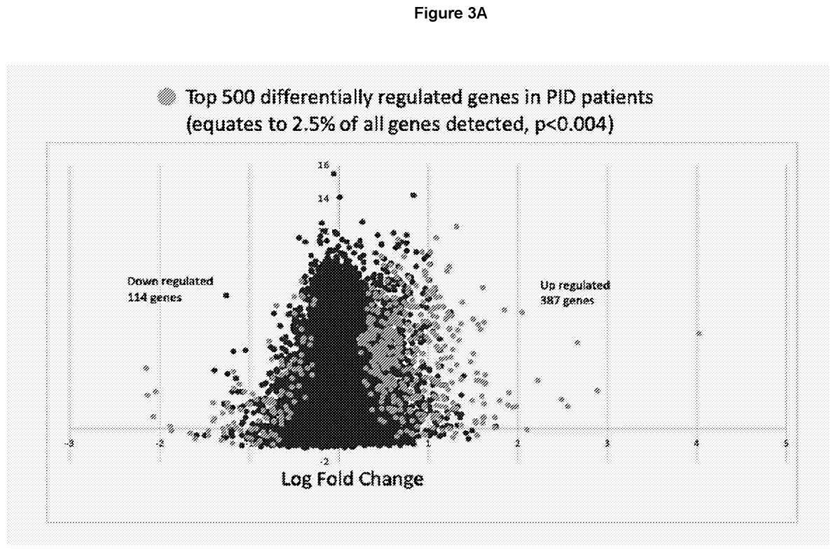

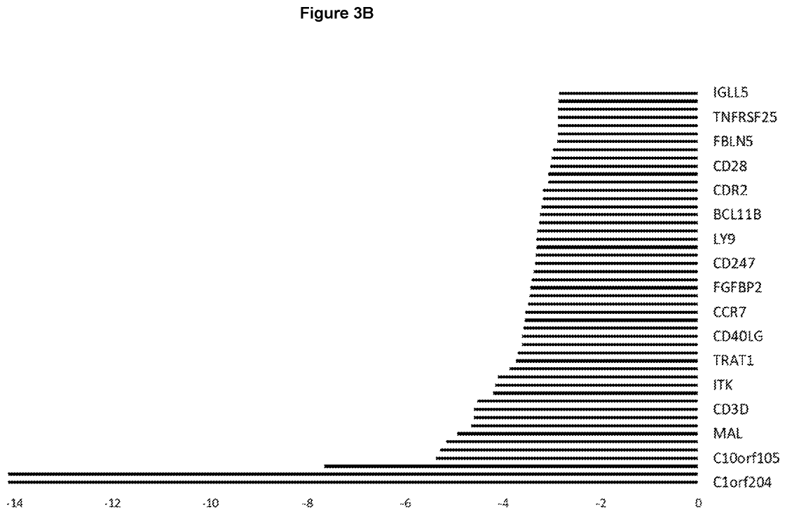

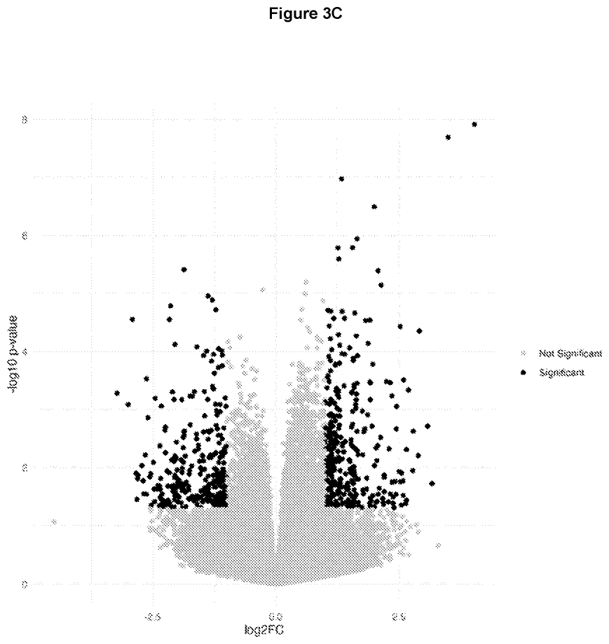

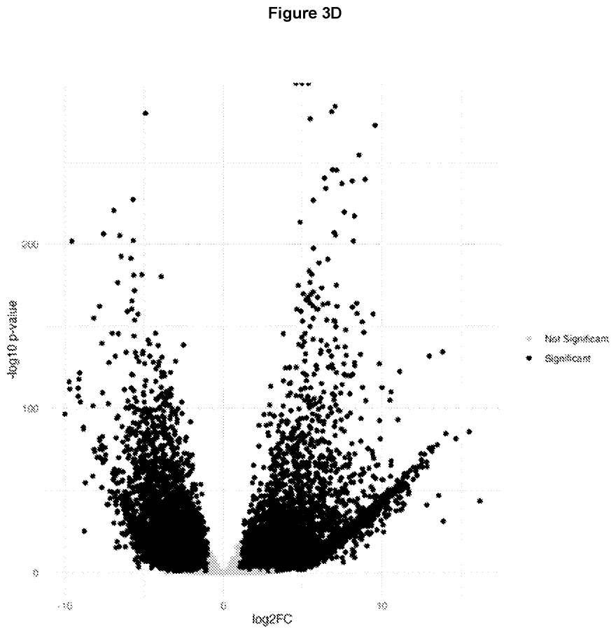

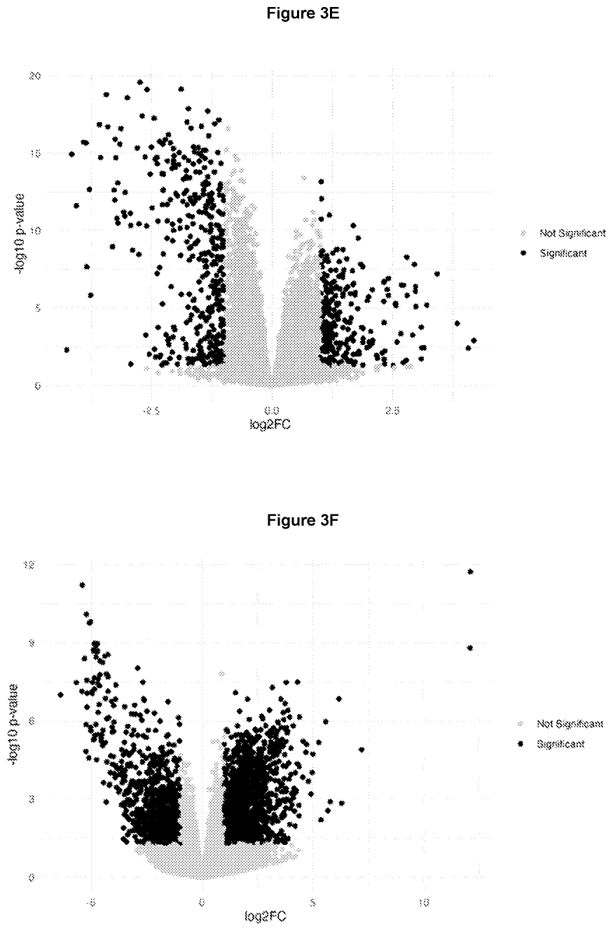

The approach to quantification was to aggregate raw counts of mapped reads using programs such as GTEx or HTSeq-count to obtain gene-level quantitation, and exon level quantification. This and similar alternatives for sequence are outlined by Conesa et al [7]. Exon read counts were retained that have an expression level of at least 2 counts per million reads (CPM) in at least one of the 20 samples. Normalization of RNA profiles adjusting for sequencing depth and other variables was performed using Bioconductor resources [8] and the EdgeR Bioconductor package [9]. FIGS. 3A& 3C shows a differential gene expression analysis comparing 19,521 genes expressed in blood of PID patients and normal matched controls. FIGS. 3D, 3E and 3F shows the differential gene expression patterns observed in patients who have SID as a consequence of HIV infection (FIG. 3D), treatment with cyclosporine, a chemotherapy drug known to cause SID (FIG. 3E) or stem cell transplantation (FIG. 3F). In all cases, there is a significant change in gene expression as a consequence of immunosuppression/immunodeficiency.

Sequence analysis for mutation detection in PID genes was performed by comparison of RNA sequence reads described above to a reference human genome or transcript reference for identification of known deleterious mutations. Paired RNA reads were aligned to genome exons using TOPHAT2 [8] and only those reads that fall within the gene exon boundaries as dictated by UCSC hg19 are used. Each set of alignments from each individual were sorted and indexed using SAMtools [10, 11]. Using the list of known or suspected PID genes and known deleterious mutations from PID discovery projects [12] and that fall within the gene exon boundaries as dictated by UCSC hg19 genome assembly, the SAMtools mpileup function (version 0.1.14) was used to extract informative allele variants in individuals. Additional approaches for variant detection in RNA sequence are becoming available [13].

RNA analysis pipelines can detect homozygous mutations using a set of PID genes and their known mutations and additionally mutations in other suspected genes [12]. Pipelines can also detect heterozygous mutations that contribute to disease phenotypes including those which are dominant mutations, or combinations of different deleterious mutations in the two alleles of the same gene [14]. In addition, RNA analysis pipelines can detect variant SNP that are in close association with causal mutations (and indicate founding mutation haplotypes) [15] that can contribute to diagnosis. In some cases, SNP variation in other parts of the genome may provide information on the likely severity of disease expression in different individuals caused by PID mutations.

### Diagnosis of PID in a Subject Using Transcriptomic Best Linear Unbiased Prediction

A prediction equation for PID diagnosis using transcriptomic BLUP was developed from a reference set of transcriptome profiles from normal and PID patients and used to create transcriptomic relationship matrices from which a predictive equation was derived. The reference set of transcriptome profiles was used to create transcriptomic relationship matrices as previously described for microbial molecular signatures [16]. A transcriptome profile is the vector of counts of sequenced reads that align to the collection of human genes (or exon) sequences in the UCSC hg19 genome or reference human transcriptome database. The reads are generated by untargeted sequencing of cDNA derived from RNA. These transcriptome profiles relate to the relative abundance of different mRNA species. The model used assumes a normal distribution, as such the transcriptome profile will be log transformed and standardised. Several transcriptome profiles were combined from an n×m matrix X with elements xᵢⱼ, the log transformed and standardised count for sample i for gene (or exon) j, with n samples and m genes. Genes with <10 reads in total aligning to them were removed from the matrix prior to standardising. These profiles were compared to make a transcriptome relationship matrix (calculated as G=XX′/m). BLUP is used to predict the disease status. A mixed model was fitted to the data: y=1ₙμ+Zg+e. Where y is the vector of disease phenotypes, with one record per sample, 1ₙ is a vector of ones, u is the overall mean, Z is a design matrix allocating records to samples, and g is a random effect estimate ˜N(0,Gσ²_{g}). The phenotypes y were corrected for other fixed effects such as age and sex prior to analysis. Using ASReml, σ²_{g} is estimated from the data and the disease status of the samples (ĝ which is a vector of length n) predicted as:

$$[ \begin{matrix}\hat{μ} \\ \hat{g}\end{matrix} ] = {[ \begin{matrix}{1}_{n}^{′} {1}_{n} & {1}_{n}^{′} Z \\ {Z}^{′} {1}_{n} & {Z}^{′} Z + {G}^{- 1} \frac{{σ}_{e}^{2}}{{σ}_{g}^{2}}\end{matrix} ]}^{- 1} [ \begin{matrix}{1}_{n}^{′} y \\ {Z}^{′} y\end{matrix} ]$$

Solving the equations results in an estimate of the mean and an estimate of the residual for each transcriptome profile, such that ĝ has the dimensions n×1. For each transcriptome profile, the predicted disease phenotype was

ĝᵢ+\hat{μ}.

Transcriptome profile prediction for PID was performed in the free R statistical software (version 3.1.2; The R Foundation for Statistical Computing; http://www.r-project.org/) and package rrBLUP was used. A transcriptome relationship matrix was fitted into BLUP and validated using two-fold cross-validation, where PID and non-PID are either training or validation sets, and an alternative procedure called leave-one-out in which one individual is removed sequentially from the dataset to estimate the disease prediction value using the remaining data. Individuals being predicted are always omitted from the training set. Tables 1 and 2 shows the list of 500 predictive variant genes used in the prediction model.

TABLE 1
List of top 500 predictive variant genes in alphabetical order (continued
next page) from both healthy control and PID subjects.

| | | | | | |
|---|---|---|---|---|---|
| ABCG2 | AC002480.2 | AC004817.4 | AC005165.1 | AC005730.1 | AC007952.6 |
| AC010615.2 | AC011379.1 | AC011444.2 | AC017099.1 | AC018755.4 | AC023301.1 |
| AC023355.1 | AC024032.2 | AC024267.1 | AC024940.1 | AC073172.1 | AC087203.2 |
| AC087481.1 | AC092490.1 | AC092802.1 | AC092821.1 | AC093909.6 | AC099489.3 |
| AC099521.3 | AC103810.5 | AC104090.1 | AC104389.2 | AC104809.2 | AC109326.1 |
| AC111000.4 | AC123912.4 | AC124312.3 | AC126544.1 | AC130366.1 | AC133919.3 |
| AC136475.5 | AC243829.1 | AC253572.1 | ACHE | ACKR1 | ADAM29 |
| ADARB2 | ADRA2A | ADRB1 | ADTRP | AGGF1P1 | AHSP |
| AJAP1 | AL008636.1 | AL008707.1 | AL031432.2 | AL031593.1 | AL121835.2 |
| AL139220.2 | AL139276.1 | AL157895.1 | AL161781.2 | AL353597.3 | AL353616.1 |
| AL353729.2 | AL356585.1 | AL391097.1 | AL590399.5 | AL592158.1 | AL645929.1 |
| AL773545.3 | ALAS2 | ALOX15 | ALOX15B | ALPK2 | ALPL |
| ANKRD20A11P | ANKRD20A4P | ANKRD22 | AOC1 | AOC3 | AP000350.2 |
| AP000350.6 | APOBEC3B | APOL4 | ARHGAP8 | ATOH8 | ATP1A4 |
| ATP1B2 | B4GALNT3 | B4GALNT4 | BATF2 | BCAM | BCL2L1 |
| BEND3P1 | BMP3 | BTNL9 | C14orf132 | C17orf99 | C19orf33 |
| C1QB | C4BPA | CA1 | CA3-AS1 | CACNG6 | CAV1 |
| CCDC144A | CCL3L3 | CCNA1 | CD177 | CD19 | CD22 |
| CDH2 | CEACAMP3 | CEROX1 | CFH | CHL1 | CHST8 |
| CICP27 | CLC | CLEC4F | CLRN1 | CMBL | CNR1 |
| CNTNAP2 | CNTNAP3 | COL19A1 | CPA3 | CPSF1P1 | CRYM |
| CSMD1 | CTNNAL1 | CTSE | CTSG | CTTNBP2 | CTXN2 |
| CXCL10 | CXCL8 | CYP4F29P | DAAM2 | DAAM2-AS1 | DAB1 |
| DACT1 | DDX11L10 | DEFA1 | DEFA3 | DEFA4 | DIPK2B |
| DLGAP1 | DMC1 | DSC1 | DSP | DUX4L9 | EDA |
| EGR1 | EIF3CL | ELAPOR1 | EPB42 | ETV7 | FAM106A |
| FAM106A | FAM153CP | FAM157A | FAM210B | FAT1 | FCRL5 |
| FCRLA | FKBP1B | FOLR3 | FREM3 | GAPDHP14 | GATA2 |
| GBP1P1 | GIMAP3P | GLDC | GPM6A | GPX1P1 | GRIK4 |
| GSTM1 | GSTM3 | GTF2H2B | GYPB | H2BP2 | HBG2 |
| HBM | HBQ1 | HDC | HEPACAM2 | HEPH | HERC2P10 |
| HLA-DQA2 | HLA-DQB1 | HLA-DQB1-AS1 | HLA-DQB2 | HLA-DRB5 | HLA-G |
| ICOSLG | IFI27 | IFI44 | IFI44L | IFIT1 | IFIT1B |
| IGF2 | IGFBP2 | IGHA1 | IGHA2 | IGHD | IGHG1 |
| IGHG2 | IGHG3 | IGHG4 | IGHM | IGHV1-2 | IGHV1-24 |
| IGHV1-3 | IGHV1-69D | IGHV2-26 | IGHV2-5 | IGHV2-70 | IGHV3-13 |
| IGHV3-15 | IGHV3-21 | IGHV3-23 | IGHV3-33 | IGHV3-48 | IGHV3-49 |
| IGHV3-53 | IGHV3-7 | IGHV3-74 | IGHV4-39 | IGHV4-4 | IGHV4-59 |
| IGHV5-10-1 | IGKC | IGKV1-12 | IGKV1-16 | IGKV1-17 | IGKV1-27 |
| IGKV1-33 | IGKV1-39 | IGKV1-5 | IGKV1-6 | IGKV1-9 | IGKV1D-33 |
| IGKV1D-39 | IGKV1D-8 | IGKV2-24 | IGKV2-30 | IGKV2D-28 | IGKV2D-29 |
| IGKV3-11 | IGKV3-15 | IGKV3-20 | IGKV4-1 | IGLC1 | IGLC2 |
| IGLC3 | IGLC7 | IGLV1-40 | IGLV1-44 | IGLV1-47 | IGLV2-8 |
| IGLV3-1 | IGLV3-19 | IGLV3-21 | IGLV3-25 | IGLV5-45 | IGLV6-57 |
| IGLV7-43 | IGLV7-46 | IGLV8-61 | IL1RL1 | IL5RA | INTS4P1 |
| IRF6 | ISG15 | ISM1 | ITGA2B | ITLN1 | JCHAIN |
| KANK2 | KAZN | KCNG1 | KCNH2 | KIAA0319 | KIR2DS4 |
| KIR3DL1 | KIR3DL2 | KLHL14 | KRT1 | KRT72 | KRT73 |
| KRT73-AS1 | LAIR2 | LARGE1 | LEP | LGSN | LINC00189 |
| LINC00570 | LINC00683 | LINC00824 | LINC01291 | LINC01293 | LINC01876 |
| LINC02073 | LINC02141 | LINC02193 | LINC02288 | LINC02289 | LINC02397 |
| LINC02458 | LINC02470 | LINC02596 | LMOD1 | LPAR3 | LPL |
| LRP1B | LRRC2 | LTF | LY6G6E | LYPD2 | MACROD2 |
| MAGI2-AS3 | MAOA | MAP7D2 | MARCO | MDGA1 | MEG3 |
| MFSD2B | MS4A2 | MT1L | MTDHP3 | MTND3P9 | MYL4 |
| MYO3B | MYOM2 | MZB1 | NAIPP3 | NBPF13P | NEBL |
| NEFL | NETO1 | NEXMIF | NF1P8 | NKX3-1 | NOG |
| NRCAM | NRXN3 | NSFP1 | NT5M | NTN4 | OCLNP1 |
| OLFM4 | OR2AK2 | OR2L9P | OR2T8 | OR2W3 | ORM1 |
| OSBP2 | OTOF | OVCH1 | OVCH1-AS1 | PAGE2B | PAQR9 |
| PAX5 | PAX8-AS1 | PAX8-AS1 | PCDHGA5 | PCDHGB2 | PDZK1IP1 |
| PGM5 | PHF24 | PI3 | PLSCR4 | PLVAP | PPP4R4 |
| PRKY | PRSS33 | PSMA6P1 | PTGES | PTGFR | PTPN20 |
| PWP2 | PXDN | RAP1GAP | RHD | RN7SL3 | RNASE3 |
| RNF182 | RNY1 | RNY3 | ROBO1 | RP11-706O15.3 | RP11-706O15.5 |
| RPL13P12 | RPL3L | RPL9P33 | RPSAP47 | RSAD2 | RUNDC3A |
| S100B | S100P | SAXO2 | SCARNA5 | SDK2 | SEC14L3 |
| SEC14L5 | SELENBP1 | SERPINB10 | SERPING1 | SGCD | SGIP1 |
| SIGLEC1 | SIGLEC12 | SIGLEC14 | SIGLEC8 | SLC12A1 | SLC2A14 |
| SLC2A4 | SLC38A11 | SLC44A4 | SLC44A5 | SLC4A1 | SLC5A4-AS1 |
| SLC6A19 | SLC6A8 | SLC6A9 | SMARCA1 | SMIM1 | SMIM24 |
| SMN2 | SMPD4P1 | SNORA23 | SNORA47 | SNORA49 | SNORA53 |
| SNORA68 | SNORA80B | SNORD3A | SNORD3B-1 | SNORD3C | SNTG2 |
| SNX18P13 | SNX18P9 | SORCS3 | SOX5 | SPP1 | SPTB |
| STOX1 | SYCP2L | TACSTD2 | TAS2R41 | TAS2R43 | TAS2R60 |
| TAS2R62P | TAS2R64P | TBC1D27P | TENM4 | TENT5C | TGM3 |
| THEGL | TMCC2 | TMEM158 | TMEM176A | TMEM176B | TMTC1 |
| TNFRSF13B | TNFRSF17 | TNR | TNS1 | TRBV30 | TRDV2 |
| TREML5P | TSIX | TSPAN7 | TSPEAR | TSPEAR-AS1 | TSPEAR-AS2 |
| TTC4P1 | TUBB2A | TUBB2B | TUBBP5 | U2AF1 | UBBP1 |
| UGT2B11 | USP32P1 | USP32P2 | VMO1 | VWCE | VWDE |
| WDR63 | WNT7A | XIST | XK | XKR3 | Y_RNA |
| ZFP57 | ZMAT4 | ZNF208 | ZNF215 | ZNF462 | ZNF727 |
| ZNF860 | ZNF890P | | | | |

TABLE 2
List of top 500 predictive variant genes in alphabetical order (continued next page) from healthy controls and
patients with immunodeficiency (including HIV infection and PID) used in creating an SID prediction model.

| | | | | | |
|---|---|---|---|---|---|
| AAMP | ABCA2 | ABCA7 | ABHD16A | ABHD17A | ABHD4 |
| ABLIM1 | ACTR1B | ADAM15 | ADIPOR1 | AGO2 | AGPAT2 |
| AHCTF1 | AHCTF1P1 | AHNAK | AKAP17A | ALAS2 | ALMS1 |
| AMPD2 | ANAPC2 | ANKRD36BP2 | ANO6 | ANXA2P2 | ANXA6 |
| APBB1 | APOL3 | APRT | AQP3 | ARAP1 | ARAP1-AS1 |
| ARF3 | ARHGAP4 | ARHGDIA | ARHGEF18 | ARL6IP4 | ASCC2 |
| ATG9A | ATP2A3 | ATP6V0C | ATP8B2 | BABAM1 | BAG6 |
| BAK1 | BCL2L1 | BCR | BIN1 | BLVRB | BNIP3L |
| BSG | BZW1 | C15orf39 | CA1 | CABIN1 | CALCOCO1 |
| CCDC117 | CCDC71 | CCDC88B | CCR2 | CCR4 | CCR6 |
| CCR7 | CD247 | CD28 | CD3E | CD4 | CD5 |
| CD68 | CDC34 | CDC37 | CENPB | CHD3 | CHI3L1 |
| CHMP1A | CHMP7 | CKAP5 | COQ8A | CORO7 | CPSF1 |
| CPVL | CRY2 | CTSD | CXXC1 | CYTH2 | DCAF12 |
| DCTN1 | DDX3Y | DDX49 | DGCR2 | DGKZP1 | DHX9 |
| DMTN | DNAH1 | DNAJB2 | DOCK2 | DOK2 | DPP7 |
| DYNLL2 | EDC4 | EEF1A1 | EEF1A1P5 | EEF1G | EEF2 |
| EGLN2 | EHBP1L1 | EIF2S3B | EIF4A1 | EIF4B | EIF4G1 |
| EIF4H | EIF6 | ENDOD1 | ENGASE | ENO1 | ENSG00000202198 |
| ENSG00000225178 | ENSG00000227766 | ENSG00000234961 | ENSG00000235105 | ENSG00000236194 | ENSG00000244491 |
| ENSG00000251095 | ENSG00000253356 | ENSG00000254873 | ENSG00000261552 | ENSG00000267484 | ENSG00000271581 |
| ENSG00000272396 | ENSG00000273149 | ENSG00000274629 | ENSG00000277654 | ENSG00000279166 | ENSG00000279369 |
| EPB41 | ESYT1 | F13A1 | FAM168B | FAM210B | FBXO7 |
| FBXW5 | FCN1 | FECH | FKBP8 | FLII | FLNA |
| FLNB | FOXO4 | FURIN | FUT7 | G6PD | GALNT4 |
| GANAB | GAPDH | GATA1 | GBGT1 | GCN1 | GEMIN4 |
| GET3 | GNB2 | GNL3L | GPR108 | GPR174 | GPRASP1 |
| GPX1 | GRINA | GSTP1 | GTF2F1 | GTF2IP4 | GUK1 |
| GYPC | GZMM | H1-2 | H1-3 | H1-4 | H1-5 |
| H2BC12 | H2BC18 | H2BC9 | H3-3A | H4C2 | H4C5 |
| H4C8 | HEMGN | HK1 | HLA-DMB | HLA-DOA | HMGN2 |
| HMGN2P5 | HNRNPC | HPS6 | HS1BP3 | IFFO2 | IFI30 |
| IGF2R | IGHA1 | IGHM | IGKC | IGKV3-20 | IGLC1 |
| IGLC2 | IGLC3 | IKBKE | IKZF3 | IL7R | INF2 |
| INO80B | INPP4B | INPPL1 | INSYN2B | INTS1 | INTS5 |
| IRF2BP1 | IRF8 | IRF9 | ITGA2B | ITGA4 | ITGA5 |
| ITGB3 | KBTBD11 | KCNA3 | KDM5C | KDM5D | KIF21B |
| KLF1 | KLHDC3 | KREMEN1 | KRT1 | KRT18P31 | LCK |
| LDB1 | LDLRAP1 | LIME1 | LINC02972 | LMF2 | LRBA |
| LRCH4 | LRP1 | LRRN3 | LSP1 | MAD1L1 | MADD |
| MAF1 | MAP4K2 | MATK | MBD3 | MBNL3 | MCM3 |
| MCM5 | MEGF6 | MFNG | MGAT5 | MICAL2 | MKRN1 |
| MPEG1 | MPP1 | MRC2 | MSN | MSNP1 | MT-ATP6 |
| MT-ATP8 | MT-CO1 | MT-CO2 | MT-CO3 | MT-CYB | MT-ND1 |
| MT-ND2 | MT-ND3 | MT-ND4 | MT-ND4L | MT-ND5 | MT-ND6 |
| MT-RNR1 | MT-RNR2 | MT-TY | MTHFS | MXD4 | MYH9 |
| NAGA | NAPSB | NBAS | NBPF14 | NBPF19 | NCF1B |
| NCOA4 | NDST1 | NDST2 | NDUFA11 | NELFB | NELL2 |
| NEURL4 | NFE2L1 | NGRN | NHSL2 | NIBAN2 | NISCH |
| NME2 | NOTCH2NLA | NUDCD3 | OBSCN | ODC1 | OGFR |
| OR2W3 | ORMDL3 | PACS1 | PBX2 | PDZD4 | PDZK1IP1 |
| PES1 | PFKL | PHOSPHO1 | PITHD1 | PLCB2 | PLEC |
| PLEKHO2 | PLXNB2 | PLXND1 | POLD4 | POLDIP3 | POLRMT |
| POMK | PPAN | PPP2R1A | PPP2R5B | PPP6R1 | PRAL |
| PRDX6 | PRKCSH | PRKY | PRPF19 | PRPF6 | PSAP |
| PSMD2 | PTGDR2 | PTMA | PUF60 | PWAR5 | PYCARD-AS1 |
| PYGB | R3HDM4 | RAB2B | RAB43 | RANGAP1 | RAPGEF6 |
| RAVER1 | RBM38 | RELL1 | RGCC | RILP | RN7SKP203 |
| RN7SKP255 | RN7SKP71 | RN7SL3 | RN7SL4P | RN7SL5P | RN7SL674P |
| RNF182 | RNPEPL1 | RNU4-1 | RNU4-2 | RPL10P16 | RPL13 |
| RPL13A | RPL13AP5 | RPL14 | RPL18 | RPL18A | RPL19 |
| RPL29 | RPL3 | RPL4 | RPL7 | RPL7A | RPL7P9 |
| RPL8 | RPLP0 | RPLP0P6 | RPS10 | RPS15 | RPS2 |
| RPS27 | RPS2P46 | RPS2P5 | RPS4Y1 | RPS6KA4 | RPSA |
| SACS | SAE1 | SASH3 | SCAP | SCARNA12 | SCARNA21 |
| SCARNA5 | SCARNA6 | SCARNA7 | SDHAF2 | SELENBP1 | SELPLG |
| SH3BP5L | SHMT2 | SIPA1 | SLC15A3 | SLC25A39 | SLC25A6 |
| SLC4A1 | SLC6A8 | SMARCD2 | SMCR8 | SMG1P3 | SMG5 |
| SNCA | SND1 | SNORA48 | SNORA63 | SNORA73A | SNORA73B |
| SNORD10 | SNORD17 | SNORD3A | SPECC1L | SPOCK2 | SPTAN1 |
| SRXN1 | ST6GALNAC4 | ST6GALNAC6 | STIL | STIMATE | STRADB |
| SYNGR2 | TAGLN2 | TAL1 | TBC1D9B | TCF7 | TCIRG1 |
| TENT5C | TESC | TFDP2 | TGFBI | TLCD4 | TLN1 |
| TM9SF1 | TMEM185B | TMEM204 | TMEM250 | TMEM35B | TMEM63B |
| TMEM65 | TMSB4X | TNFRSF10C | TNFSF13 | TNS1 | TNS3 |
| TOMM6 | TRANK1 | TRAPPC9 | TRBC1 | TRBC2 | TREML2 |
| TRIM28 | TRIM58 | TSIX | TSSC4 | TUBB4B | TUBGCP6 |
| TWF2 | TXLNGY | TXNDC5 | UBASH3A | UBC | UBE2V1 |
| UBL7 | UNC119 | UQCRC1 | USP12 | USP5 | USP9Y |
| UTY | VCP | VPS51 | WBP1 | WDR81 | WDTC1 |
| XAB2 | XIST | XK | XRCC6 | YBX1 | YBX1P1 |
| ZAP70 | ZBED1 | ZBED6 | ZBTB7B | ZC3H11A | ZER1 |
| ZGPAT | ZNF134 | ZNF316 | ZNF460 | ZNF672 | ZNF792 |
| ZNF805 | ZNF831 | | | | |

Increasing the transcriptome sample reference numbers from affected and unaffected individuals facilitates additional training for the transcriptomic BLUP and iteratively increases accuracy of prediction and diagnosis.

TABLE 3
List of top 500 differentially regulated genes in alphabetical order (continued
next page) in subjects with PID compared to healthy controls.

| | | | | | |
|---|---|---|---|---|---|
| ABCC13 | AC004987.9 | AC007365.3 | AC023590.1 | AC026271.5 | AC092580.4 |
| ACER3 | ACHE | ACKR1 | ACP1 | ACVR1C | ADAM9 |
| ADIPOR1 | AHSP | AKAP6 | ALAS2 | AMPD2 | ANKRD22 |
| ANP32B | ANXA2 | ANXA2P2 | AP2M1 | AP2S1 | APOL3 |
| APOL4 | APOLD1 | APOPT1 | ASCL2 | ASNA1 | ATP5B |
| ATP5E | ATP5J2 | ATP6V0C | AURKA | BAMBI | BCAM |
| BCAS4 | BEND5 | BISPR | BLVRA | BSCL2 | BSG |
| BST2 | BTF3 | BTNL9 | C16orf74 | C17orf99 | C18orf8 |
| C2 | CA1 | CALCOCO2 | CAPG | CAPNS1 | CARD16 |
| CARM1 | CASP1 | CASP7 | CBX7 | CCL5 | CCRL2 |
| CD177 | CD200 | CD33 | CD36 | CD3EAP | CD68 |
| CD8A | CDAN1 | CDC34 | CDCA7 | CDKL1 | CEBPA |
| CERK | CETP | CFAP45 | CHMP4B | CHPT1 | CISD2 |
| CITED2 | CLDN5 | CLEC11A | CLEC1B | CLEC6A | CLEC9A |
| CMPK2 | CNN3 | CNPPD1 | CNTLN | COA6 | COCH |
| COL27A1 | COL4A3 | COL6A1 | COMT | COX5A | COX6B1 |
| CPNE8 | CREG1 | CROCCP2 | CRYM | CSTB | CTB-193M12.5 |
| CTD-2002H8.2 | CTD-2319I12.10 | CTD-2540L5.6 | CTD-2619J13.14 | CTD-3252C9.4 | CTNNAL1 |
| CTSB | CXCL10 | CYB5A | CYBB | CYTH2 | DAAM2 |
| DCP1B | DDIAS | DDX60 | DESI1 | DHX58 | DLGAP1 |
| DLGAP5 | DPCD | DPM2 | DRAP1 | DTL | DYNLL1 |
| DYNLRB1 | E2F1 | EFCAB2 | EIF2AK2 | EIF4EBP1 | EIF5AL1 |
| EMC3 | EMID1 | ENC1 | EOMES | EPB41L3 | EPOR |
| EPSTI1 | ETV7 | FADS1 | FAHD1 | FAM104A | FAM104B |
| FAM132B | FAM177B | FAM210B | FAT4 | FBLN2 | FBXO6 |
| FCER1G | FCGR1C | FGFR1OP | FIS1 | FKBP1B | FKBP8 |
| FRMD3 | FTL | FTLP3 | FUCA1 | FUNDC2 | GABARAP |
| GABARAPL2 | GALM | GBP1 | GBP1P1 | GBP5 | GCNT1P3 |
| GLRX5 | GNAS | GNG7 | GOLGA8R | GP9 | GPD2 |
| GPR137B | GPR146 | GPR150 | GPR61 | GPR84 | GPS2 |
| GPX1 | GPX4 | GRIK4 | GSPT1 | GSTK1 | GYPC |
| HBA1 | HBA2 | HBG2 | HBM | HCST | HDGF |
| HDX | HERC5 | HIST1H2BN | HP | HPS1 | HSPB1 |
| IDH1 | IDH2 | IFI27 | IFI27L2 | IFI35 | IFI44 |
| IFI44L | IFI6 | IFIT1 | IFIT1B | IFIT3 | IFITM1 |
| IGF2 | IGHA2 | IGHG2 | IGHG4 | IGHV2-26 | IGKV1-8 |
| IGKV1D-8 | IGLV5-45 | IGSF9 | IL15 | IL15RA | IL1RAP |
| INTS12 | ISG15 | ITSN1 | JAK2 | JHDM1D-AS1 | JUND |
| KCNH2 | KCNH8 | KCNK5 | KEL | KRT1 | KRT72 |
| KRT73 | KRT73-AS1 | LAP3 | LDLR | LGALS3 | LHFPL2 |
| LINC00534 | LINGO2 | LPXN | LRRC8A | LSM12P1 | LY6E |
| LYL1 | MAF1 | MARCO | MASTL | MBNL1-AS1 | MCOLN1 |
| MCOLN2 | MED16 | MEG3 | METTL9 | MFSD2B | MGST1 |
| MIIP | MPP1 | MRPS17P5 | MSC-AS1 | MSMO1 | MT1E |
| MT1F | MT1L | MT2A | MTCO2P11 | MTCO3P11 | MTHFD1 |
| MX1 | MYBL2 | MYOM2 | NAPA | NDUFA5P11 | NDUFAF3 |
| NDUFB8P2 | NDUFS7 | NDUFV3 | NEIL1 | NEIL3 | NRG1 |
| NRIR | NT5C3A | NT5E | NT5M | NTAN1 | NUCB1 |
| NUDT14 | NUDT19P5 | OAS1 | OAS2 | OAS3 | OASL |
| OGN | OLA1P1 | OST4 | PA2G4 | PAIP2B | PAQR9 |
| PARP12 | PARP14 | PARP9 | PARPBP | PBXIP1 | PCBP3 |
| PCED1A | PCTP | PDE3B | PDE6A | PDK4 | PFDN2 |
| PHACTR1 | PHF11 | PHF24 | PIGC | PLA2G7 | PLEK2 |
| PLK3 | PLOD2 | PNP | PQLC1 | PRDX2 | PRR5 |
| PSEN2 | PSMB2 | PSME1 | PSME2 | PSTPIP2 | PTTG1 |
| RAB39A | RAP1GAP | RASGRP2 | RASSF6 | RBM11 | RBX1 |
| RETN | REXO2 | RFX2 | RGS10 | RHOU | RILP |
| RN7SKP296 | RN7SL128P | RN7SL4P | RNA5SP202 | RNF187 | RNY1 |
| RP1-167A14.2 | RP1-257A7.4 | RP11-102N12.3 | RP11-103B5.4 | RP11-1193F23.1 | RP11-12A2.1 |
| RP11-153M7.3 | RP11-158I9.5 | RP11-162A23.5 | RP11-20D14.6 | RP11-288L9.4 | RP11-305L7.1 |
| RP11-403B2.7 | RP11-422P24.12 | RP11-466G12.3 | RP11-474P2.4 | RP11-500G10.5 | RP11-609D21.3 |
| RP11-61I13.3 | RP11-676J12.9 | RP11-68I3.5 | RP11-70C1.1 | RP11-713P17.3 | RP11-77H9.8 |
| RP11-798G7.6 | RP11-7F17.3 | RP11-81H14.2 | RP11-886P16.10 | RP11-96K19.4 | RP4-641G12.3 |
| RP5-1028K7.2 | RP5-998N21.4 | RPA4 | RPS27P16 | RPSAP6 | RSAD2 |
| RSRC1 | RSU1 | RTP4 | RUFY4 | RUNDC3A | S100A12 |
| S100A4 | SAMD9L | SAP30 | SCD | SDC2 | SDSL |
| SELENBP1 | SELK | SERF2 | SERPINB9 | SERPING1 | SERTAD2 |
| SESTD1 | SGIP1 | SHARPIN | SHC3 | SHTN1 | SIAH2 |
| SIGLEC1 | SKA3 | SLC1A3 | SLC30A4 | SLC6A19 | SLC9A7 |
| SLFN11 | SLFN12 | SMIM1 | SMIM24 | SNTB1 | SNX15 |
| SNX3 | SP100 | SPATS2L | SPHK1 | SPOCD1 | SPRY1 |
| SPSB2 | SQLE | ST13P6 | STAT1 | STAT2 | STIL |
| STK11 | STOM | STYK1 | SVBP | SWAP70 | SYBU |
| TAGLN2 | TCN2 | TCTN1 | TDRD6 | TESC | TFEC |
| TFR2 | THOC7 | THRA | TICAM2 | TLR7 | TM7SF2 |
| TMEM158 | TMSB4XP8 | TNFSF13 | TNNT1 | TOMM6 | TOR1A |
| TPGS1 | TPGS2 | TPM1 | TRBV7-6 | TRBV7-9 | TRBV9 |
| TRIM22 | TRNP1 | TSC22D3 | TSPAN17 | TSPO2 | TSTA3 |
| TTC9 | TUBBP5 | TXNL4A | TYMP | UBA52 | UBAP2 |
| UBB | UBBP4 | UBE2F | UBE2L6 | UBL7 | UGT8 |
| USP18 | UST-AS1 | VCAN | VRK2 | WARS | WASF4P |
| XAF1 | XKRX | XXyac-YR38GF2.1 | YARS | YBX1P10 | ZBP1 |
| ZDHHC23 | ZDHHC4P1 | ZMAT2 | ZNF662 | ZNF677 | ZNF711 |
| ZNF772 | ZNRF1 | | | | |

TABLE 4
List of top 500 differentially regulated genes in alphabetical order (continued next page)
from an SID model including healthy controls, patients with HIV infection and PID.

| | | | | | |
|---|---|---|---|---|---|
| ABCB9 | ABHD12B | ABRAXAS1 | ACAT2 | ACO1 | ACSBG1 |
| AGBL5 | AHSA2P | AKAP13-AS1 | AMPH | ANKDD1A | ANKRD19P |
| ANKRD23 | ANKS1B | AP4M1 | APOBEC2 | APTR | ARAP1-AS1 |
| ARG2 | ARGFXP2 | ARHGEF39 | ASCC1 | ATAD5 | ATG10 |
| ATP11A-AS1 | ATP6V1C2 | AZI2 | B3GAT2 | B3GNT4 | BAZ1A-AS1 |
| BCDIN3D-AS1 | BEST1 | BRD7 | BRSK2 | BTG1-DT | C19orf44 |
| C1orf112 | C1orf56 | C1orf74 | C5orf58 | CABP7 | CACNB2 |
| CALHM6-AS1 | CALML4 | CAPN12 | CBX3P3 | CCDC73 | CCDC85C |
| CCL3-AS1 | CCNA2 | CCNE2 | CDK4 | CEBPB-AS1 | CENPO |
| CENPP | CEP164P1 | CEPT1 | CFAP36 | CFLAR-AS1 | CHKA |
| CINP | CLEC1B | CLMN | CMC1 | CMTM7 | CNNM2 |
| CNPY2-AS1 | CNTD1 | COA1 | COA5 | COG1 | CORO1A-AS1 |
| COX20 | CP | CRIM1 | CSPP1 | CTTN-DT | CXCR5 |
| CYREN | DCBLD2 | DDN-AS1 | DDTL | DEGS2 | DLEC1 |
| DNAAF8 | DNAH17 | DNAI7 | DOCK8-AS1 | DUSP14 | DUSP28 |
| EFCAB15P | EHBP1-AS1 | EIF3J | EMC4 | EMC9 | EME1 |
| EME2 | EML4-AS1 | ENO3 | ENSG00000183308 | ENSG00000188242 | ENSG00000197813 |
| ENSG00000206028 | ENSG00000215022 | ENSG00000219410 | ENSG00000225335 | ENSG00000225339 | ENSG00000225864 |
| ENSG00000225963 | ENSG00000226849 | ENSG00000227218 | ENSG00000227766 | ENSG00000227782 | ENSG00000228113 |
| ENSG00000228318 | ENSG00000228634 | ENSG00000228863 | ENSG00000228925 | ENSG00000230521 | ENSG00000230532 |
| ENSG00000231760 | ENSG00000232295 | ENSG00000232807 | ENSG00000233223 | ENSG00000233340 | ENSG00000233937 |
| ENSG00000234961 | ENSG00000235027 | ENSG00000235945 | ENSG00000236213 | ENSG00000236540 | ENSG00000236986 |
| ENSG00000237126 | ENSG00000237798 | ENSG00000237938 | ENSG00000238246 | ENSG00000240535 | ENSG00000241666 |
| ENSG00000243423 | ENSG00000243797 | ENSG00000244491 | ENSG00000245156 | ENSG00000245748 | ENSG00000247903 |
| ENSG00000248544 | ENSG00000248571 | ENSG00000251002 | ENSG00000251095 | ENSG00000253356 | ENSG00000254263 |
| ENSG00000254814 | ENSG00000254873 | ENSG00000255557 | ENSG00000256116 | ENSG00000256249 | ENSG00000257279 |
| ENSG00000257452 | ENSG00000258017 | ENSG00000258044 | ENSG00000258365 | ENSG00000258424 | ENSG00000258428 |
| ENSG00000258666 | ENSG00000258682 | ENSG00000258745 | ENSG00000258843 | ENSG00000258891 | ENSG00000258928 |
| ENSG00000258959 | ENSG00000259407 | ENSG00000259483 | ENSG00000259536 | ENSG00000259544 | ENSG00000259792 |
| ENSG00000259972 | ENSG00000260005 | ENSG00000260249 | ENSG00000260304 | ENSG00000260465 | ENSG00000260744 |
| ENSG00000260891 | ENSG00000261267 | ENSG00000261346 | ENSG00000261460 | ENSG00000261505 | ENSG00000261552 |
| ENSG00000262049 | ENSG00000262777 | ENSG00000263272 | ENSG00000263466 | ENSG00000264304 | ENSG00000264577 |
| ENSG00000265749 | ENSG00000265907 | ENSG00000266490 | ENSG00000267484 | ENSG00000267724 | ENSG00000268583 |
| ENSG00000268618 | ENSG00000269072 | ENSG00000269243 | ENSG00000269318 | ENSG00000269982 | ENSG00000271380 |
| ENSG00000271581 | ENSG00000271821 | ENSG00000271980 | ENSG00000272144 | ENSG00000272211 | ENSG00000272396 |
| ENSG00000273149 | ENSG00000273218 | ENSG00000273261 | ENSG00000273489 | ENSG00000273599 | ENSG00000274292 |
| ENSG00000274425 | ENSG00000274629 | ENSG00000275888 | ENSG00000277299 | ENSG00000277978 | ENSG00000278330 |
| ENSG00000278765 | ENSG00000278993 | ENSG00000279159 | ENSG00000279179 | ENSG00000279344 | ENSG00000279412 |
| ENSG00000279434 | ENSG00000279641 | ENSG00000279689 | ENSG00000279722 | ENSG00000279748 | ENSG00000280007 |
| ENSG00000280067 | ENSG00000280143 | ENSG00000280194 | ENSG00000280195 | ENSG00000280211 | ENSG00000280295 |
| ENTPD1-AS1 | EOGT-DT | EP400P1 | EPB41L4A | EPM2A-DT | ETFBKMT |
| EXOSC8 | FAM118B | FAM161B | FAM177A1 | FAM184B | FANCA |
| FANCI | FANCL | FARP1 | FRMD5 | FSD2 | GADD45GIP1 |
| GAS5-AS1 | GATC | GCNT7 | GDF15 | GEMIN2 | GEMIN7-AS1 |
| GFM2 | GNAL | GNPTG | GPR37L1 | GRIN3B | GRIP2 |
| GSN-AS1 | GTF2IRD1P1 | H1-12P | H2AZ1-DT | H2BC20P | H4C9 |
| HCG25 | HDGFL3 | HEATR1 | HEXIM2-AS1 | HLA-DQB1-AS1 | HNRNPA1P76 |
| HSD17B1 | IAH1 | ICAM4-AS1 | IFI6 | IL10RB-DT | IL24 |
| IRAK1BP1 | JAG1 | JMJD8 | JSRP1 | KCNMB3 | KIF24 |
| KLC1-AS1 | KLHDC7B-DT | KMO | KPNB1-DT | KRR1 | LACTB2-AS1 |
| LDHAL6A | LIG3 | LINC01531 | LIPA | LIPE-AS1 | LNX1 |
| LOXL3 | LRRC37A16P | LRRC51 | LRRC75A | LYSMD1 | LYSMD4 |
| MAK16 | MALSU1 | MAP3K12 | MAP3K20-AS1 | MCF2L2 | MCM3AP-AS1 |
| MCPH1-AS1 | MED11 | MED12L | METTL5 | MFSD11 | MIF-AS1 |
| MIR302CHG | MIR497HG | MLLT11 | MMACHC | MME-AS1 | MPI |
| MRNIP | MRPL53P1 | MRPS18C | MRTFA-AS1 | MSH6 | MT-TY |
| MTCO3P43 | MTND5P28 | MVP-DT | MYCBP2-AS1 | MYH11 | MYL12-AS1 |
| MYL5 | NAB2 | NANS | NCF4-AS1 | NEIL1 | NFATC2IP-AS1 |
| NHSL1 | NKX6-2 | NLRP7 | NME9 | NMI | NOP16 |
| NRG4 | NSA2P1 | NUDCD2 | NUDT17 | OSGEPL1 | PAPLN |
| PAQR3 | PCBP1-AS1 | PDAP1 | PEX10 | PGLS-DT | PHACTR1 |
| PHKA2-AS1 | PIGB | PIGHP1 | PIK3CD-AS1 | PINK1-AS | PITX1-AS1 |
| PLCG1-AS1 | PLEKHH1 | PLLP | PLTP | PMFBP1 | PODXL |
| PPM1F-AS1 | PRAL | PRG4 | PRMT5-AS1 | PRPH2 | PRR29 |
| PRSS53 | PSMA3 | PSMB8-AS1 | PSMC3IP | PSMD6 | PTCD1 |
| PTPMT1 | PXN-AS1 | PYCARD-AS1 | RAD52 | RCN1P2 | RFC5 |
| RIPOR3 | RNF175 | RNF39 | RNF43 | RPA2 | RPL13AP5 |
| RPL30-AS1 | RSPH9 | RUFY2 | RUSC1-AS1 | SCAND2P | SCUBE3 |
| SDR39U1 | SEMA3F-AS1 | SETD6 | SH3BP5-AS1 | SHMT1 | SIAE |
| SIAH2-AS1 | SLA2 | SLC12A5-AS1 | SLC22A5 | SLC35E4 | SLC35F2 |
| SLC46A1 | SLC51A | SLC5A10 | SLC5A2 | SLC7A6OS | SLC9A3R1-AS1 |
| SLC9B1 | SLIRP | SMTNL1 | SNX21 | SNX22 | SNX32 |
| SPAG5-AS1 | SPATA1 | SPATA21 | SRR | SRRD | SRSF12 |
| SSBP3-AS1 | STARD5 | SUV39H2 | SYCP3 | SYNM-AS1 | TAF9BP1 |
| TAPT1-AS1 | TATDN1 | TCHP | TEAD2 | TET2-AS1 | TIAM2 |
| TLDC2 | TMC5 | TMEM120B | TMEM132A | TMEM184A | TMEM200B |
| TMEM220 | TMEM223 | TMEM50B | TMEM97 | TP5313 | TRABD-AS1 |
| TRGV2 | TRGV4 | TRIM13 | TRIM59 | TRNT1 | TRPT1 |
| TSIX | TSPAN16 | TVP23A | TXNDC17 | UBXN11 | UQCC2 |
| USP3-AS1 | USP50 | VAC14-AS1 | VCAN-AS1 | VIM-AS1 | VPS39-DT |
| WDR5B-DT | WDR73 | WDR74 | WDR83 | WNT2B | XAF1 |
| XRRA1 | ZACN | ZNF225-AS1 | ZNF333 | ZNF337-AS1 | ZNF385C |
| ZNF451-AS1 | ZNF561-AS1 | ZNF594-DT | ZNF710-AS1 | ZNF80 | ZNF814 |
| ZNRD1ASP | ZRANB2-AS1 | | | | |

RNAseq can provide information on the status of the immune system in individuals indicative of immune status that could be evidence of a predisposition to SID, or acquisition of SID

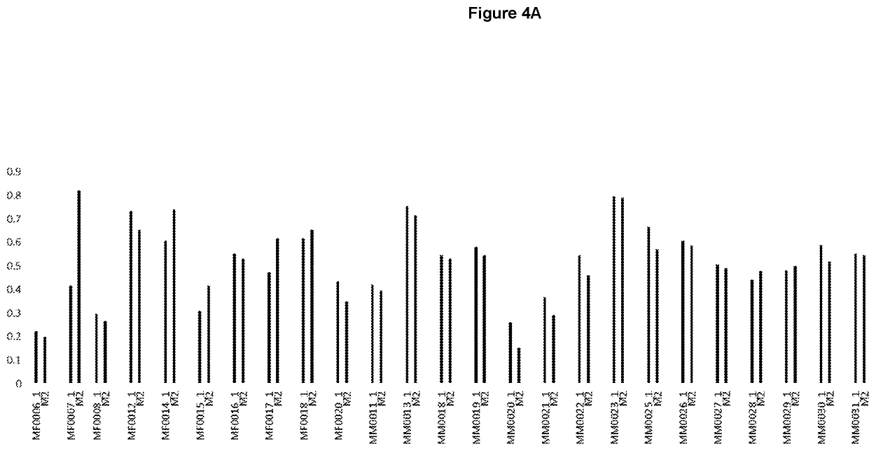

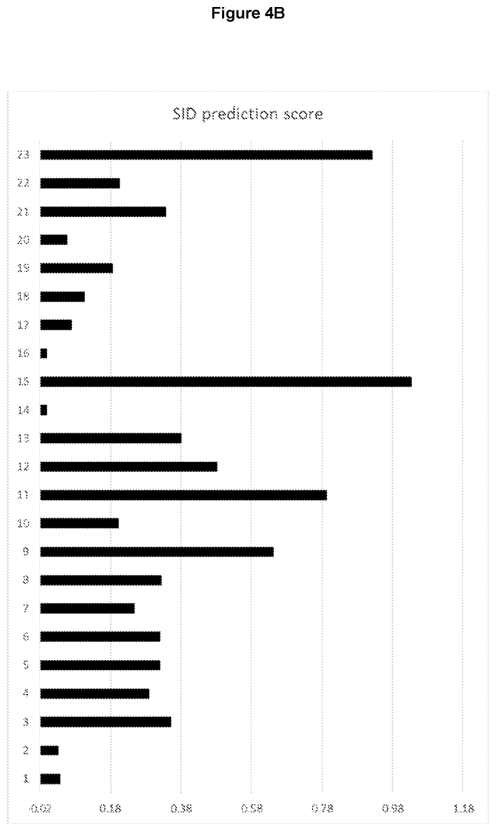

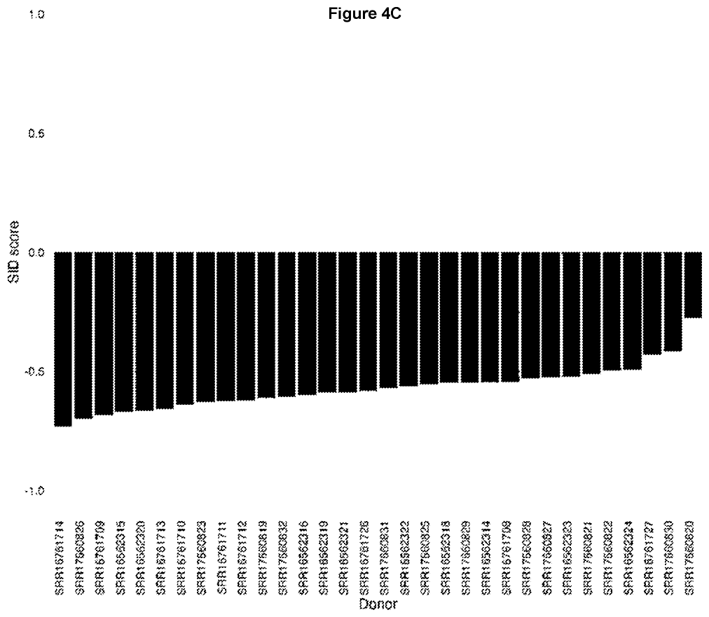

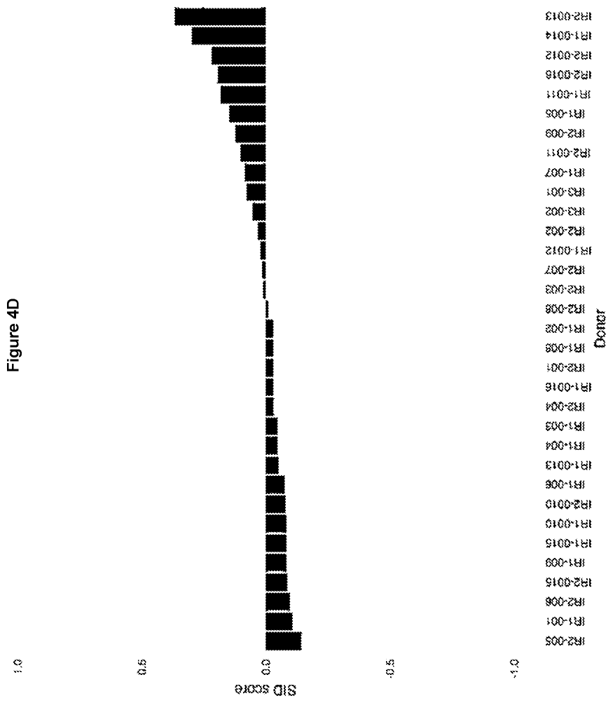

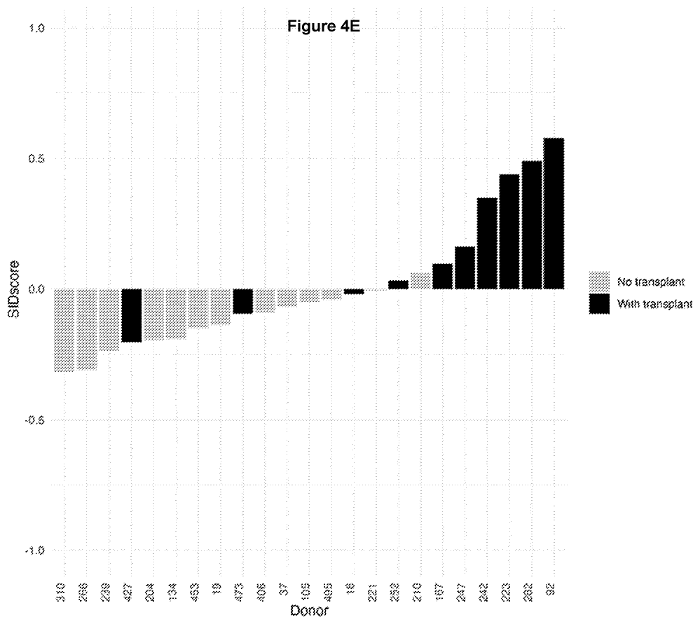

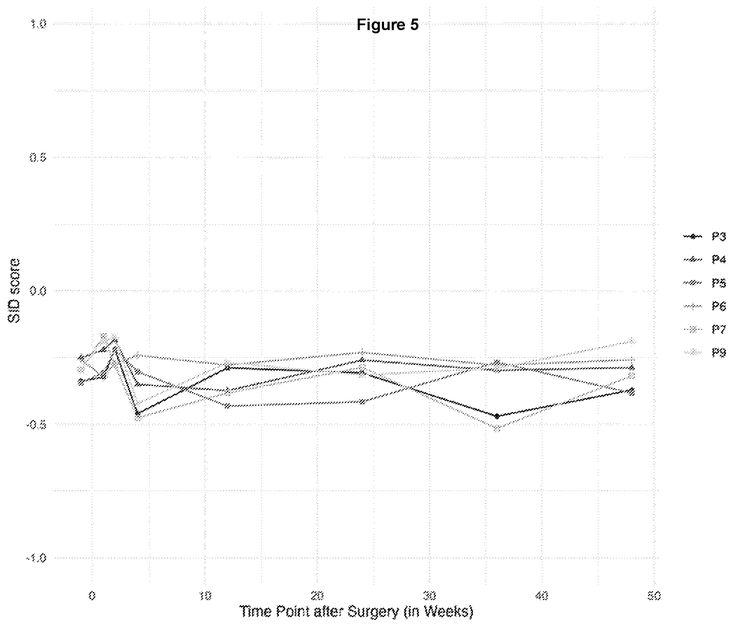

RNAseq can provide information on the status of the immune system in individuals indicative of immune status that could be evidence of a predisposition to SID, or acquisition of SID. FIG. 3F shows in an example of the impact of transplantation therapy on the blood RNAseq profile which is concomitant with an impact on the immune system, SID and predisposition to infection. After three months of treatment with cyclosporin hundreds of gene expression changes are observed, and 50 of the most suppressed genes are graphed as fold change down regulation in FIG. 3E. Using a larger number RNAseq profiles of individuals untreated and with therapy with immune suppressing drugs (used in transplantation), or exposure to other factors, a BLUP model can be created using genes identified in Tables 1 to 4 (and if required additional genes identified in analysis such as in FIG. 3B-F) that provides a score that can predict the susceptibility of an individual to SID upon a given treatment and monitor the development and onset of SID longitudinally with a treatment in a clinical setting. An example of such an application is shown in FIG. 4A-B and FIG. 5, noting these are individuals in a study not specifically identified as having immune health issues.

Application of a predictive SID model to evaluate SID susceptibility score on a set of individuals derived from their whole blood RNAseq expression profiles.

RNAseq expression signatures can be used as a measure of susceptibility to SID but also development of SID. RNAseq can provide information on the status of the immune system in individuals indicative of immune status that could be evidence of a predisposition to SID, or acquisition of SID. FIG. 4A-E show an example of application of a BLUP model (built from batch-corrected training data from a combination of patients with immunodeficiency (HIV infection and/or PID) and normal healthy controls. The model is then applied to a set of patients that may or may not develop SID and predisposition to infection. For example, in FIG. 4E, the SID prediction model is applied to patients who have undergone immunosuppressant therapy and hematopoietic stem cell transplant. The model can accurately detect patients who have received a transplant.

Additionally, as shown in FIG. 4A-B, diabetic patients were treated with metformin in a study by Ustinova et al., (Ustinova et al. PLOS one, 2020; 15(8):e0237400) and in general the patients ranged in their overall susceptibility scores (noting diabetes is a risk factor for SID), and in most cases this score was not affected by metformin over the drug treatment (metformin is not expected to induce SID not being an immune suppressant) except in patient MF0007, who was the only patient to develop significantly low IgA levels. Using a larger number RNAseq profiles of individuals untreated and with therapy with immune suppressing drugs (used in transplantation), or exposure to other factors, a BLUP model can be further developed that provides an improved prediction or diagnosis score that can a) predict the susceptibility of an individual to SID upon a given treatment AND b) monitor the development and onset of SID longitudinally with a treatment in a clinical setting. An example of a longitudinal application is shown in FIG. 4A-B and FIG. 5 where susceptibility scores are calculated for patients at different times following percutaneous osseointegrated lower limb implantation. In this example, the patients are not immunosuppressed and have normal immune systems.

The analysis demonstrates an example of variation in immune status of individuals where a high score may indicate weaker immune systems and therefore susceptibility to SID or sub-clinical SID under different therapy or treatment circumstances. FIGS. 4B & 4D shows specific examples where application of a predictive SID model to evaluate SID susceptibility score on a set of individuals in an immunosuppression and bone marrow transplantation trial. The analysis demonstrates an example of variation in immune status of individuals where a high score may indicate weaker immune systems and therefore susceptibility to SID under in this case corticosteroid therapy or other treatment circumstances.

### Metagenome Profile Determination

Untargeted massively parallel sequencing of ribosomal or microbial DNA was performed to generate reference PID metagenome profiles.

### i) Sample Collection and DNA Extraction

For microbiome profile acquisition, DNA was extracted from buccal swabs and hair follicles using DNA extraction kits as described below.

### Buccal Swap Sample Collection:

1. Teeth are brushed sometime within the 4 hours before sampling with eating avoided (following teeth brushing) prior to sampling.

2. The cotton swab or nylon brush was used to push and swipe the buccal mucosa.

3. Cotton was stripped from the swab with sterile tweezers and placed into a tube containing lysis buffer or the head of the nylon brush was placed directly into a tube containing lysis buffer.

Extracting DNA from Buccal Swabs

### Materials:

1. QIAamp DNA Mini Kit (Qiagen, Cat. Cat No./ID: 51304, Cat No./ID: 51306)

2. RNase A solution (R6148-25 ml, Sigma)

3. Preparation of lysis buffer: 20 mg lysozyme in the solution of 25 mM Tris. HCl, pH8.0; 2.5 mM EDTA, pH8.0 and 1% Triton X-100

### Protocol:

Place buccal swab (cotton) in a 1.5 mL or 2 mL tube.
Add 400 μl lysis buffer (20 mg/ml lysozyme in the solution of 25 mM Tris. HCl, pH 8.0; 2.5 mM EDTA, pH8.0 and 1% Triton X-100). Mixed by pushing cotton several time and pipetting.
Incubate at 37° C. for 60 min.
Add 40 μl proteinase K (20 mg/ml) and 400 μl Buffer AL. Mix thoroughly by vortex for 10 seconds. (Note: do not mix proteinase K directly to Buffer AL.) Briefly centrifuge the tube to remove drops from inside the lid.
Incubate at 55° C. for 60 min. Vortex occasionally during incubation to disperse the sample.
Incubate further 15 min at 80° C. to inactivate the proteinase K.
Remove the solution to a new tube. Push tightly cotton using a pipette tip and remove the solution as possible.
Add RNase A solution (R6148-25 ml, Sigma) 8 μl, 37° C. for 60 min.
Add 450 μl ethanol (96-100%) to the sample and mix by pulse-vortexing for 15 s. Briefly centrifuge the tube to remove drops from inside the lid.
Apply the mixture (including all the precipitate, need to divide mixture into 2 portions) to the Mini spin column. Close the cap and centrifuge at maximum speed for 1 min.
Add 500 μl Buffer AW1 to the column. Close the cap and centrifuge at maximum speed for 1 min and discard the filtrate.
Add 500 μl Buffer AW2 to the column. Close the cap and centrifuge at maximum speed for 1 min and discard the filtrate.
Transfer the column to new 2 ml collection tube and centrifuge at maximum speed for 2 min.
Put column in a 1.5 ml tube and add 50-100 μl Buffer EB (Qiagen) or 10 mM Tris-HCl, pH 8.5, depending on gDNA concentration required. Incubate at room temperature for 1-3 min and then centrifuge at maximum speed for 2 min.

For skin sampling, skin preparation instructions include avoiding bathing and avoiding emollients or antimicrobial soaps or shampoos for 12 hours prior to all sampling. Sampling sites include the retroauricular crease, cubital fossa or volar forearm. From a 4 cm² area, bacterial swabs (via Epicentre swabs) and scrapes (via sterile disposable surgical blade) are obtained and incubated in an enzymatic lysis buffer and lysozyme as described above for buccal swab samples.

Amplification of Microbial DNA from Buccal Swabs

### PCR 16S PCR

Primers used: 341F/806R primers which cover 16S V4 region. The primer sites are targeted by the “forward” primer 341F and the “reverse” primer 806R. In addition, bar coding primers for Illumina MySeq sequencing are included (shaded below).

Illumina Multiplexing Read1 Sequencing
Primer was added (806R Ad)
(SEQ ID NO: 1)
5′ [symbol]
GGACTACHVGGGTWTCTAAT3′
Illumina Multiplexing Read2 Sequencing
Primer was added (341F Ad)
(SEQ ID NO: 2)

[symbol]

CCTACGGGAGGCAGCAG 3′

TABLE 5
PCR reaction set up: 50 μl reaction

| Component | Final conc. | Vol (μl) |
|---|---|---|
| 5 × Phusion HF buffer | 1× | 10 |
| 10 mM dNTP | 0.2 mM each | 1 |
| Primer mix (10 μM each) | 0.1 μM each | 0.5 |
| DMSO | 5% | 2.5 |
| Phusion DNA polymerase | 0.02 U/μl | 0.5 |
| Water | | 33.5 |
| gDNA Template | | 2 |
| Total | | 50 |

TABLE 6
Thermal cycler conditions

| Temp. | 98° C. | 98° C. | 60° C. | 72° C. | 72° C. |
|---|---|---|---|---|---|
| Time | 30 sec | 10 sec | 15 sec | 15 sec | 1 min |

| | | | |
|---|---|---|---|
| Cycle | 1 | 30 | 1 |

Sequencing of Microbial DNA from Buccal Swabs

Standard Illumina protocols used for sequencing MiSeq.

### ii) Targeted and Untargeted Massively Parallel Sequencing

Library preparation for sequencing was performed using indexing protocol using Illumina barcoding primers as described by the manufacturer. The indexes are a short third read of the sequencing run. Briefly, DNA is sheared to 300 bp, adapters are added by ligation, and then indexes added using PCR. The libraries are then quantified and pooled. Paired-end sequencing of genomic DNA was performed on a HiSeq2000™ sequencer. Sequence reads were trimmed so that the average Phred quality score for each read will be above 20. If the read length is below 50 after trimming, the read was discarded.

### Ill) Metagenome Profile Analysis

### Diagnosis of SID in a Subject Using Metagenomic Best Linear Unbiased Prediction

The reference set of metagenome profiles generated were used to create metagenomic relationship matrices essentially as previously described [16]. A metagenome profile is the vector of counts of sequenced reads that align to a collection of 16S rRNA sequences or other available or generated reference sequence sets (here referred to as contigs) in a database. The reads were generated by untargeted sequencing of microbial DNA, or by sequencing 16S ribosomal sequences amplified by PCR from microbial DNA. These metagenome profiles relate to the relative abundance of different microbial species. The model used assumes a normal distribution, as such the metagenome profile will be log transformed and standardised.

Several metagenomic profiles were combined from an n×m matrix X with elements xᵢⱼ, the log transformed and standardised count for sample i for contig j, with n samples and m contigs. Contigs with <10 reads in total aligning to them will be removed from the matrix prior to standardising. These profiles are compared to make a microbiome relationship matrix (calculated as G=XX′/m). Best linear unbiased prediction was used to predict the phenotype. A mixed model was fitted to the data: y=1ₙμ+Zg+e. Where y is the vector of clinical phenotype, with one record per sample, 1ₙ is a vector of ones, u is the overall mean, Z is a design matrix allocating records to samples, and g is a random effect estimate ˜N(0,Gσ²_{g}). Using ASReml, σ²_{g} was estimated from the data and the phenotypes of the samples (ĝ which is a vector of length n) predicted as:

$$[ \begin{matrix}\hat{μ} \\ \hat{g}\end{matrix} ] = {[ \begin{matrix}{1}_{n}^{′} {1}_{n} & {1}_{n}^{′} Z \\ {Z}^{′} {1}_{n} & {Z}^{′} Z + {G}^{- 1} \frac{{σ}_{e}^{2}}{{σ}_{g}^{2}}\end{matrix} ]}^{- 1} [ \begin{matrix}{1}_{n}^{′} y \\ {Z}^{′} y\end{matrix} ]$$

Solving the equations results in an estimate of the mean and an estimate of the residual for each metagenome profile, such that ĝ has the dimensions n×1. For each metagenome profile, the predicted phenotype is ĝᵢ+\hat{μ}.

Microbiome profile prediction for SID was performed in the free R statistical software (version 3.1.2; The R Foundation for Statistical Computing; http://www.r-project.org/) and package rrBLUP were used. A metagenomics relationship matrix was fitted into best linear regression model (BLUP) and validated using two-fold cross-validation, where SID and non-SID are either training or validation sets, and an alternative procedure called leave-one-out in which one individual is removed sequentially from the dataset to estimate the disease prediction value using the remaining data. Individuals being predicted were always omitted from the training set.

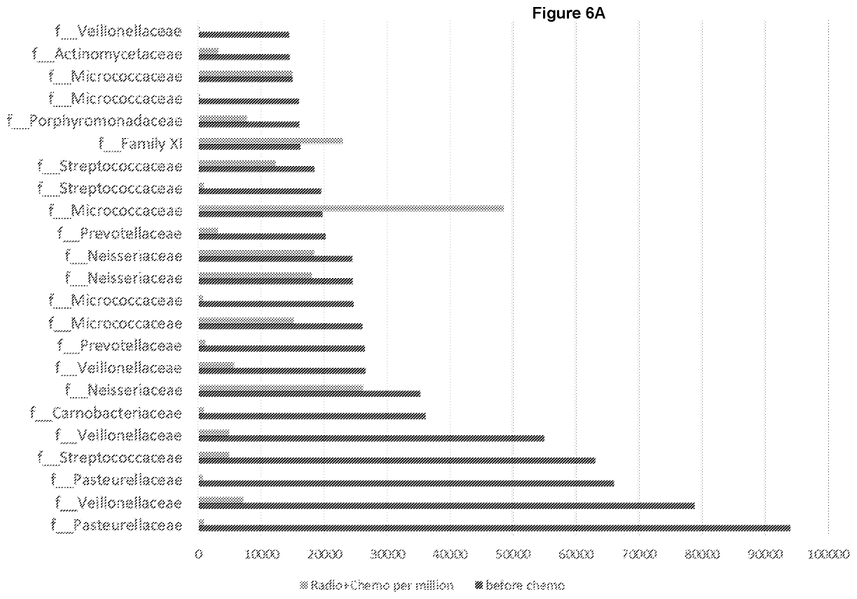

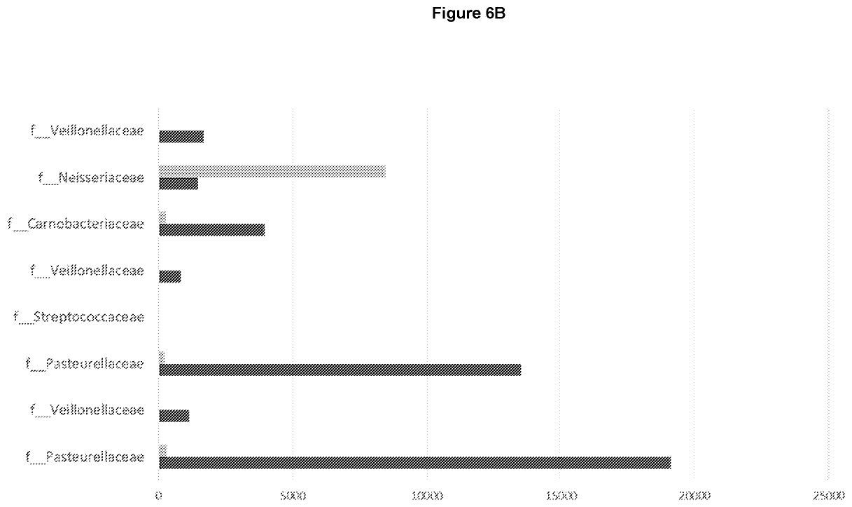

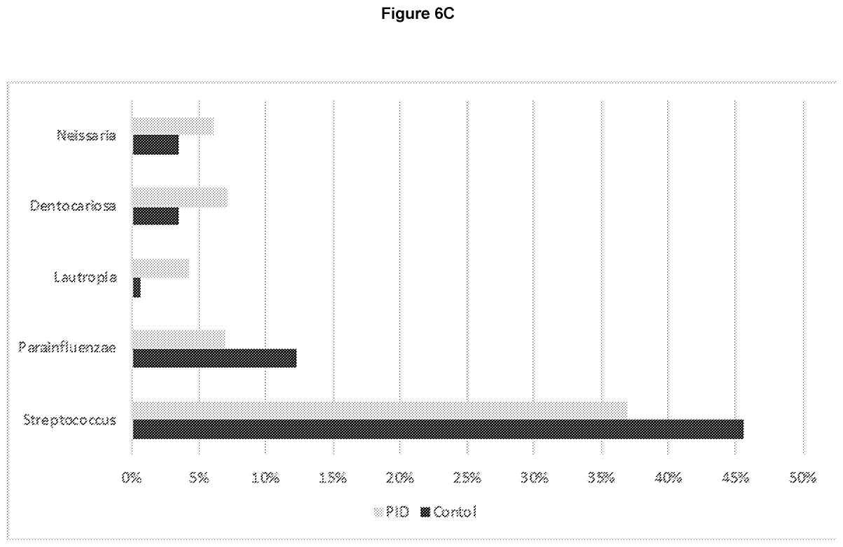

Data used was from supplementary data provided by Kumpitsch et al (Kumpitsch et al. Scientific reports, 2020; 10:16582). 16S or whole genome metagenomic sequencing of microbial populations can provide information on the status of microbial balance in individuals that is indicative of immune status that could be evidence of a predisposition to or acquisition of SID. FIG. 6A and FIG. 6B shows in an example of the impact of chemotherapy and radiotherapy on the mouth microbiome which is concomitant with an impact on the immune system, SID and predisposition to infection. FIG. 6C shown an example of differences in buccal microbial diversity in patients diagnosed with PID compared to healthy controls. Using a larger number of mouth or buccal microbiome profiles from individuals untreated and with chemotherapy (used in cancer) or immune suppressing drugs (used in transplantation), or exposure to other factors, a BLUP model can be created that provides a score that can: a) predict the susceptibility of an individual to SID upon a given treatment AND b) monitor the development and onset of SID longitudinally with a treatment in a clinical setting.

FIG. 6A shows an analysis demonstrating example of significant differences in specific bacterial populations in the mouth microbiome of a group of patients before (dark bars) and after (light bars) undergoing combined radiotherapy and chemotherapy (cisplatin). Combined data from 6 patients. FIG. 6B shows Analysis demonstrating example of changes over time in specific bacterial populations in the mouth microbiome of a patient before (dark bars) and after (light bars) undergoing 45 days of combined radiotherapy and chemotherapy according to the example of the disclosure.

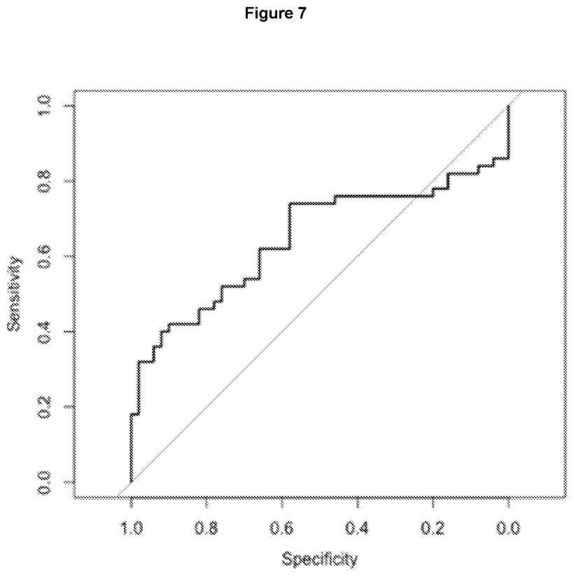

FIG. 7 is an ROC curve for individual patient prediction scores generated using microbiome profile prediction for SID. In this example, the prediction model is trained on and applied to immunodeficient patients with PID and normal individuals. The microbial prediction model can be applied to other examples of immunodeficiency such as patients treated with chemotherapy, radiotherapy or immunosuppressive drugs where treatment changes immune regulation of microbial diversity.

### Diagnosis of PID in a Subject by Combined RNA and Metagenomic Best Linear Unbiased Prediction

Integrative (transcriptomics and metagenomics) prediction was performed in R statistical software. Twenty positive and 20 negative diagnoses for PID and blood transcriptomic profile and metagenomic profile were fitted into a linear regression model.

An extended relationship matrix was developed that combines the Z matrix described above for RNA transcript abundances with the metagenomic Z¹ relationship matrix as follows:

$$y = {1}_{n} μ + {Z}_{g} + {Z}^{1} {g}^{1} + e$$

The coefficients in the output were multiplied with blood transcriptomic profile and metagenomic profile respectively to calculate the integrative predicted PID disease phenotype. Accuracy of prediction was assessed by Pearson's correlation, ‘r’, that is, the correlation between the measured values with predicted values.

The results demonstrated that: firstly, transcriptome profiles were able to predict PID in these circumstances; secondly, integrating transcriptomic with metagenomics information can increase prediction accuracy. Updating the transcriptome and microbiome sample reference for training with affected and unaffected individuals will increase prediction accuracy.

### Diagnosis of SID in a Subject by Gene Sequence Based Prediction

Genomic variants in mRNA such as SNP variants that are in linkage with defective immune genes in the population may be useful for prediction. A known mutation in mRNA may be inferred or imputed from linkage to a co-occurring haplotype marker in the mRNA expressed from the same gene, or nearby gene on the chromosome. This genomic information can be obtained from genomic sequence or RNA sequence and used to inform diagnosis alone or in combination with transcriptomic BLUP or transcriptomic BayesR.

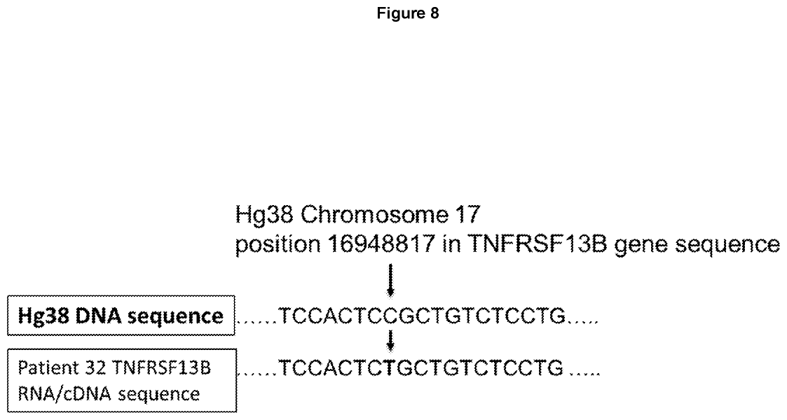

SNPs in mRNA sequences acquired through RNAseq can provide genotype information on haplotypes in individuals (in a similar manner to SNP chips, or whole genome, or whole exome sequencing) indicative of immune traits (such as low lymphocytes) that could predispose individuals to SID. FIG. 8 shows in an example of how RNA derived sequence can be used to detect a base difference in one gene TACI or TNFRSF13B known to predispose individuals to immunodeficiency (https://pubmed.ncbi.nlm.nih.gov/24074872/), and other gene variants that are a determinant of immune cells numbers in published GWAS studies (Orru et al. Cell, 2013; 155(1):242-56) and may also predispose individuals to immunodeficiency (Rudilla et al Expanding the Clinical and Genetic Spectra of Primary Immunodeficiency-Related Disorders With Clinical Exome Sequencing: Expected and Unexpected Findings.)

Many SNPs or variants associated with immune traits (up to thousands) could be identified in RNAseq data that are linked or predictive of immune traits. These SNPs either in individual key genes or combinations of SNPs with small effects, can be used to complement information from with RNAseq expression signatures, to improve the measure of susceptibility to SID. RNAseq expression signatures can be used as a measure of susceptibility, but also development of SID and active SID.

### Diagnosis of SID in a Subject by Different Linear Mixed Model Approaches

Alternative linear mixed model approaches to BLUP such as BayesR applied to genomic prediction based on across genome sequence variation as described by Kemper et al [20] can also be applied to transcriptomic and/or metagenomics data in the same way as has been described above using BLUP, with the X matrix describing individuals by normalised read counts per contig. The BayesR method assumes that the true effects of gene expression are derived from a series of normal distributions, the first with zero variance, up to one with moderate to large variance. The advantage of BayesR over BLUP is that the effects of individual genes are not compressed as hard towards the mean as in BLUP. BayesR approaches can also be extended as described by Macleod et al [21] to include known biological information (BayesRC) such as immune system regulatory function.

### Diagnosis of SID in a Subject by Machine Learning Approaches

Alternative approaches to linear mixed models may also be applied to transcriptomic and/or metagenomic data in a predictive way similar to what has been described above using BLUP to enable classification and prediction of SID. Machine learning, support vector machines, and neural networks can provide an alternate approach to linear mixed models for using transcriptomic and/or metagenomic data from patients and normal controls as input for patient classification, and subsequently predictive model training. A similar approach has been used for classifying cancer patients into high or low risk groups and for the development of predictive models to assist prognosis [22], and using tumour RNAseq data as input for this purpose is being investigated [23].

The linear mixed models may be coupled with a descriptive report from the subject that would be useful for an informed clinician to assist with assessment of immune system dysregulation, cells and pathways effected, disease status and perhaps preferred treatment.

To do this genes significantly differentially expressed in a given SID patient (for example, 20 or 50 or even 100 DE genes) can be identified, and this DE gene set subject to a pathway over-representation analysis (or Gene Set Enrichment Analysis), such as or similar to DAVID or Reactome programs and from them a report generated on the pathway and cellular functions that are affected in the subject.

A further qualitative report to supplement diagnosis could be to provide a clustering report of how the subjects transcriptome compares to other patients in the database. This can be based on the transcriptomic relationship matrix or other analysis of the differential gene expression from that patient. It would be understood that patients with mutations in the same gene cluster closer together based on their transcriptome. Once a larger database of patients is available, this clustering may assist in categorisation of newly diagnosed patients into SID disease subtype based on transcriptome (to complement and mutation detection similarities found).

### REFERENCES

1. Salem S, Langlais D, Lefebvre F, Bourque G, Bigley V, Haniffa M, Casanova J L, Burk D, Berghuis A, Butler K M et al: Functional characterization of the human dendritic cell immunodeficiency associated with the IRF8(K108E) mutation. Blood 2014, 124(12):1894-1904.
2. Naik S, Bouladoux N, Wilhelm C, Molloy M J, Salcedo R, Kastenmuller W, Deming C, Quinones M, Koo L, Conlan S et al: Compartmentalized control of skin immunity by resident commensals. Science 2012, 337(6098):1115-1119.
3. Oh J, Freeman A F, Park M, Sokolic R, Candotti F, Holland S M, Segre J A, Kong H H: The altered landscape of the human skin microbiome in patients with primary immunodeficiencies. Genome research 2013, 23(12):2103-2114.
4. Gallo V, Dotta L, Giardino G, Cirillo E, Lougaris V, D'Assante R, Prandini A, Consolini R, Farrow E G, Thiffault I et al: Diagnostics of Primary Immunodeficiencies through Next-Generation Sequencing. Frontiers in immunology 2016, 7:466.
5. Erbe M, Hayes B J, Matukumalli L K, Goswami S, Bowman P J, Reich C M, Mason B A, Goddard M E: Improving accuracy of genomic predictions within and between dairy cattle breeds with imputed high-density single nucleotide polymorphism panels. Journal of dairy science 2012, 95(7):4114-4129.
6. Zhang W, Yu Y, Hertwig F, Thierry-Mieg J, Thierry-Mieg D, Wang J, Furlanello C, Devanarayan V, Cheng J, Deng Y et al: Comparison of RNA-seq and microarray-based models for clinical endpoint prediction. Genome biology 2015, 16:133.
7. Conesa A, Madrigal P, Tarazona S, Gomez-Cabrero D, Cervera A, McPherson A, Szczesniak M W, Gaffney D J, Elo L L, Zhang X et al: A survey of best practices for RNA-seq data analysis. Genome biology 2016, 17:13.
8. Huber W, Carey V J, Gentleman R, Anders S, Carlson M, Carvalho B S, Bravo H C, Davis S, Gatto L, Girke T et al: Orchestrating high-throughput genomic analysis with Bioconductor. Nature methods 2015, 12(2):115-121.
9. Robinson M D, McCarthy D J, Smyth G K: edgeR: a Bioconductor package for differential expression analysis of digital gene expression data. Bioinformatics 2010, 26(1):139-140.
10. Etherington G J, Ramirez-Gonzalez R H, MacLean D: bio-samtools 2: a package for analysis and visualization of sequence and alignment data with SAMtools in Ruby. Bioinformatics 2015, 31(15):2565-2567.
11. Li H, Handsaker B, Wysoker A, Fennell T, Ruan J, Homer N, Marth G, Abecasis G, Durbin R: The Sequence Alignment/Map format and SAMtools. Bioinformatics 2009, 25(16):2078-2079.
12. Itan Y, Casanova J L: Novel primary immunodeficiency candidate genes predicted by the human gene connectome. Frontiers in immunology 2015, 6:142.
13. Sheng Q, Zhao S, Li C I, Shyr Y, Guo Y: Practicability of detecting somatic point mutation from RNA high throughput sequencing data. Genomics 2016, 107(5):163-169.
14. Lionakis M S: Genetic Susceptibility to Fungal Infections in Humans. Current fungal infection reports 2012, 6(1):11-22.
15. Hsu A P, Sampaio E P, Khan J, Calvo K R, Lemieux J E, Patel S Y, Frucht D M, Vinh D C, Auth R D, Freeman A F et al: Mutations in GATA2 are associated with the autosomal dominant and sporadic monocytopenia and mycobacterial infection (MonoMAC) syndrome. Blood 2011, 118(10):2653-2655.
16. Ross E M, Moate P J, Marett L C, Cocks B G, Hayes B J: Metagenomic predictions: from microbiome to complex health and environmental phenotypes in humans and cattle. PloS one 2013, 8(9):e73056.
17. Endelman J B: Ridge Regression and Other Kernels for Genomic Selection with R Package rrBLUP. The Plant Genome 2011, 4(3):250-255.
20. Kemper K E, Reich C M, Bowman P J, Vander Jagt C J, Chamberlain A J, Mason B A, Hayes B J, Goddard M E: Improved precision of QTL mapping using a nonlinear Bayesian method in a multi-breed population leads to greater accuracy of across-breed genomic predictions. Genetics, selection, evolution: GSE 2015, 47:29.
21. Macleod I M, Bowman P J, Vander Jagt C J, Haile-Mariam M, Kemper K E, Chamberlain A J, Schrooten C, Hayes B J, Goddard M E: Exploiting biological priors and sequence variants enhances QTL discovery and genomic prediction of complex traits. BMC genomics 2016, 17:144.
22. Kourou K, Exarchos T P, Exarchos K P, Karamouzis M V, Fotiadis D I Machine learning applications in cancer prognosis and prediction. Comput Struct Biotechnol J. 2014 Nov. 15; 13:8-17.
23. Han H, Liu Y. Transcriptome marker diagnostics using big data. IET Syst Biol. 2016 February; 10(1):41-8.
24. Miller A, Jeyapalina1 S, Agarwal1 J, Mansel M, Peter Beck J. A preliminary, observational study using whole-blood RNA sequencing reveals differential expression of inflammatory and bone markers post-implantation of percutaneous osseointegrated prostheses. PLoS One. 2022; 17(5):e0268977.
25. Verma S, Du P, Nakanjako P, Hermans S, Briggs J, Nakiyingi L Ellner J, Manabe Y, Salgame P. Tuberculosis in advanced HIV infection is associated with increased expression of IFNγ and its downstream targets. BMS Infect Dis. 2018; 18:220
26. Englert J, Cho M H, Lamb A E, Shumyatcher M, Barragan-Bradford D, Basil M C, Higuera A, Isabelle C, Pinilla Vera M, Dieffenbach P B, Fredenburgh L E, Kang J B, Bhatt A S, Antin J H, Ho V T, Soiffer R J, Howrylak J A, Himes B E, Baron R M. Whole blood RNA sequencing reveals a unique transcriptomic profile in patients with ARDS following hematopoietic stem cell transplantation. Respir Res. 2019; 20: 15.

1. A method for determining whether a subject has or is susceptible to developing a secondary immunodeficiency (SID), the method comprising using a linear mixed model to fit a transcriptome profile of the subject to a SID prediction equation developed by fitting into a linear mixed model a transcriptomic relationship matrix generated from a reference set of transcriptome profiles of reference subjects with and without SID, wherein the prediction equation's result indicates whether the subject has or is susceptible to SID.

2. A method for developing a secondary immunodeficiency (SID) prediction equation for determining whether a subject has or is susceptible to developing a SID, the method comprising fitting into a linear mixed model a transcriptomic relationship matrix generated from a reference set of transcriptome profiles of reference subjects with and without SID to develop the SID prediction equation.

3. The method of claim 1 or 2, further comprises measuring the transcriptome profile of the subject.

4. The method of any one of claims 1 to 3, further comprising measuring the transcriptome profiles of the reference subjects.

5. A method for the longitudinal monitoring of a secondary immunodeficiency (SID) in a subject, the method comprising using a linear mixed model to evaluate a transcriptome profile of the subject to a SID prediction equation developed by fitting into a linear mixed model a transcriptomic relationship matrix generated from a reference set transcriptome profile(s) with and without SID, wherein the prediction equation's result indicates a change to the subjects SID status.

6. A method for developing a secondary immunodeficiency (SID) prediction equation for the longitudinal monitoring of a secondary immunodeficiency (SID) in a subject, the method comprising fitting into a linear mixed model a transcriptomic relationship matrix generated from a reference set of longitudinal transcriptome profiles.

7. The method of claim 5 or claim 6, further comprises measuring the transcriptome profile of the subject:
at the start of monitoring to generate a reference set transcriptome profile, and
at any subsequent time point after an exposure event (e.g. exposure to an agent as described herein) or risk factor that could give rise to SID or in the routine monitoring of SID.

8. The method of any one of claims 1 to 7, wherein the linear mixed model is best linear unbiased prediction (BLUP), BayesR, or machine learning approaches.

9. The method of any one of claims 1 to 8, wherein the reference set further comprises a RNA sequence mutation profile.

10. The method of any one of claims 1 to 9, further comprising measuring a RNA sequence mutation profile of the subject for whom the determination of SID or susceptibility to SID is to be made.

11. The method of any one of claims 1 to 10, wherein the reference set further comprises a RNA sequence mutation profile and the linear mixed model is used to fit the transcriptome profile and a RNA sequence mutation profile of the subject to the SID prediction equation.

12. The method of any one of claims 1 to 11, wherein the reference set further comprises a DNA sequence mutation profile.

13. The method of any one of claims 1 to 12, further comprises measuring or determining the DNA sequence mutation profile of the subject for whom the determination of SID or susceptibility to SID is to be made

14. The method of any one of claims 1 to 13, wherein the reference set further comprises a DNA sequence mutation profile and the linear mixed model is used to fit the transcriptome profile and a DNA sequence mutation profile of the subject to the SID prediction equation.

15. The method of any one of claims 9 to 14, wherein the mutation profile comprises:
a) a RNA sequence of a SID gene comprising a known mutation associated with a SID;
b) a new mutation, optionally a frameshift mutation, stop codon or amino acid change, that affects structure or function of a protein encoded by a known gene mutation associated with a SID;
c) a dominant mutation in one allele associated with a SID;
d) two different mutations in the same gene, but on two different alleles that associate with a SID;
e) a known mutation in RNA that is inferred or imputed by linkage to a co-occurring marker for a mutation associated with a SID;
f) absence of expression of a gene normally expressed in non-SID subjects indicating a regulatory defect or destabilising mutation;
g) a defective exon structure indicating a splicing defect;
h) one or more, optionally one to three, additional mutations associated with a SID; or
i) a sequence of more than one other gene, or an imputed sequence of more than one other gene, that associates with SID severity.

16. The method of any one of claims 1 to 15, wherein the reference set further comprises a metagenome profile.

17. The method of any one of claims 1 to 16, further comprises measuring or determining the metagenome profile of the subject for whom the determination of SID or susceptibility to SID is to be made.

18. The method of any one of claims 1 to 16, further comprises measuring or determining the metagenome profile of the subject for whom the longitudinal monitoring of SID is to be made.

19. The method of any one of claims 1 to 16, wherein the reference set further comprises a metagenome profile and the linear mixed model is used to fit the transcriptome profile and a metagenome profile of the subject to the SID prediction equation.

20. The method of any one of claims 1 to 19, wherein the transcriptome profile or sequence mutation profile is obtained from sputum, blood, amniotic fluid, plasma, semen, bone marrow, tissue, urine, peritoneal fluid, or pleural fluid, optionally obtained by fine needle biopsy.

21. The method of claim 20, wherein the blood comprises peripheral blood mononuclear cells.

22. The method of any one of claims 16 to 21, wherein the metagenome profile is obtained from a mouth swab, nose swab, throat swab, saliva, faeces, or skin.

23. The method of any one of claims 1 to 22, wherein the subject is human.

24. The method of any one of claims 1 to 23, wherein the profile of the subject for whom the determination of SID, susceptibility to SID or monitoring of SID is to be made is determined or measured from analysing a biological sample previously obtained from the subject.

25. A computer-implemented method for processing genomic information, the genomic information comprising a subject transcriptome profile, the method comprising:
accessing a reference set of transcriptome profiles of reference subjects, each reference subject either having or not having a secondary immunodeficiency (SID);
generating a transcriptomic relationship matrix from the reference set of transcriptome profiles;
fitting the transcriptomic relationship matrix into a linear mixed model to generate a SID prediction equation; and
fitting the subject transcriptome profile to the SID prediction equation.

26. A computer-implemented method for generating a secondary immunodeficiency (SID) prediction equation, the method comprising:
accessing a reference set of transcriptome profiles of reference subjects, each reference subject either having or not having a secondary immunodeficiency (SID);
generating a transcriptomic relationship matrix from the reference set of transcriptome profiles; and
fitting the transcriptomic relationship matrix into a linear mixed model to generate the SID prediction equation.

27. A computer-implemented method for processing genomic information, the genomic information comprising a subject transcriptome profile, the method comprising:
accessing a reference set transcriptome profile of the subject for whom the monitoring of SID is to be made;
generating a transcriptomic relationship matrix from the reference set transcriptome profile;
fitting the transcriptomic relationship matrix into a linear mixed model to generate a SID prediction equation; and
fitting the subject transcriptome profile to the SID prediction equation.

28. A computer-implemented method for generating a secondary immunodeficiency (SID) prediction equation, the method comprising:
accessing a reference set transcriptome profile of the reference subject for whom the monitoring of SID is to be made;
generating a transcriptomic relationship matrix from the reference set transcriptome profile; and
fitting the transcriptomic relationship matrix into a linear mixed model to generate the SID prediction equation.

29. The computer-implemented method of any one of claims 25 to 28, further comprises measuring the transcriptome profile of the subject.

30. The computer-implemented method of any one of claims 25 to 29, further comprising measuring the transcriptome profiles of the reference subjects.

31. The computer-implemented method of any one of claims 25 to 30, wherein the linear mixed model is best linear unbiased prediction (BLUP), BayesR, random forest or machine learning approaches.

32. The computer-implemented method of any one of claims 25 to 31, wherein the reference set further comprises a RNA sequence mutation profile.

33. The computer-implemented method of claim 25 or claim 27, wherein the reference set further comprises a RNA sequence mutation profile and the linear mixed model is used to fit the transcriptome profile and a RNA sequence mutation profile of the subject to the SID prediction equation.

34. The computer-implemented method of any one of claims 25 to 33, wherein the reference set further comprises a DNA sequence mutation profile.

35. The computer-implemented method of any one of claims 25 to 34, wherein the reference set further comprises a DNA sequence mutation profile and the linear mixed model is used to fit the transcriptome profile and a DNA sequence mutation profile of the subject to the SID prediction equation.

36. The computer-implemented method of any one of claims 25 to 35, wherein the reference set further comprises a metagenome profile.

37. The computer-implemented method of claims 25 to 35, wherein the reference set further comprises a metagenome profile and the linear mixed model is used to fit the transcriptome profile and a metagenome profile of the subject to the SID prediction equation.

38. A non-transitory computer-readable medium storing instructions, which when executed by a processor cause the processor to:
access a reference set of transcriptome profiles of reference subjects, each reference subject either having or not having a secondary immunodeficiency (SID);
generate a transcriptomic relationship matrix from the reference set of transcriptome profiles;
fit the transcriptomic relationship matrix into a linear mixed model to generate a SID prediction equation;
receive a subject transcriptome profile; and
fit the subject transcriptome profile to the SID prediction equation.

39. A non-transitory computer-readable medium storing instructions, which when executed by a processor cause the processor to:
access a reference set of transcriptome profiles of reference subjects, each reference subject either having or not having a secondary immunodeficiency (SID);
generate a transcriptomic relationship matrix from the reference set of transcriptome profiles; and
fit the transcriptomic relationship matrix into a linear mixed model to generate the SID prediction equation.

40. A non-transitory computer-readable medium storing instructions, which when executed by a processor cause the processor to:
access a reference set transcriptome profile of the subject for whom the monitoring of SID is to be made;
generate a transcriptomic relationship matrix from the reference set transcriptome profile;
fit the transcriptomic relationship matrix into a linear mixed model to generate a SID prediction equation;
receive the subject transcriptome profile; and
fit the subject transcriptome profile to the SID prediction equation.

41. A non-transitory computer-readable medium storing instructions, which when executed by a processor cause the processor to:
access a reference set transcriptome profile of the subject for whom the monitoring of SID is to be made;
generate a transcriptomic relationship matrix from the reference set transcriptome profile; and
fit the transcriptomic relationship matrix into a linear mixed model to generate the SID prediction equation.

42. The non-transitory computer-readable medium storing instructions of any one of claims 38 to 41, wherein the linear mixed model is best linear unbiased prediction (BLUP), BayesR, random forest or machine learning approaches.

43. The non-transitory computer-readable medium storing instructions of any one of claims 38 to 42, wherein the reference set further comprises a RNA sequence mutation profile.

44. The non-transitory computer-readable medium storing instructions of claim 38 or claim 40, wherein the reference set further comprises a RNA sequence mutation profile and the linear mixed model is used to fit the transcriptome profile and a RNA sequence mutation profile of the subject to the SID prediction equation.

45. The non-transitory computer-readable medium storing instructions of any one of claims 38 to 44, wherein the reference set further comprises a DNA sequence mutation profile.

46. The non-transitory computer-readable medium storing instructions of any one of claims 38 to 44, wherein the reference set further comprises a DNA sequence mutation profile and the linear mixed model is used to fit the transcriptome profile and a DNA sequence mutation profile of the subject to the SID prediction equation.

47. The non-transitory computer-readable medium storing instructions of any one of claims 38 to 46, wherein the reference set further comprises a metagenome profile.

48. The non-transitory computer-readable medium storing instructions of claims 38 to 47, wherein the reference set further comprises a metagenome profile and the linear mixed model is used to fit the transcriptome profile and a metagenome profile of the subject to the SID prediction equation.

49. A method for longitudinal monitoring of SID in a subject, the method comprising using a linear mixed model to fit a metagenomics profile of the subject to a SID prediction equation developed by fitting into a linear mixed model a metagenomic relationship matrix generated from a reference set metagenomic profile of the subject for whom the monitoring of SID is to be made, wherein the prediction equation's result indicates whether the subject has a change in SID status.

50. A method for developing a secondary immunodeficiency (SID) prediction equation for the longitudinal monitoring of a secondary immunodeficiency (SID) in a subject, the method comprising fitting into a linear mixed model a metagenomic relationship matrix generated from a reference set metagenomic profile of the subject for whom the monitoring of SID is to be made, wherein the prediction equation's result indicates whether the subject has a change in SID status.

51. The method of claim 49 or claim 50, wherein the metagenome profile is obtained from a mouth swab, nose swab, throat swab or saliva.

52. The method of any one of claims 49 to 51, wherein the subject is human.

53. The method of any one of claims 48 to 52, wherein the profile of the subject for whom the monitoring of SID is to be made is determined or measured from analysing a biological sample previously obtained from the subject.


SEQUENCE LISTING (4 sequences)

SEQ ID NO: 1 - RNA, 53 nt, synthetic construct
  source 1..53: mol_type = other RNA
acactctttccctacacgacgctcttccgatctggactachvgggtwtctaat

SEQ ID NO: 2 - RNA, 51 nt, synthetic construct
  source 1..51: mol_type = other RNA
gtgactggagttcagacgtgtgctcttccgatctcctacgggaggcagcag

SEQ ID NO: 3 - DNA, 19 bp, Homo sapiens
  source 1..19: mol_type = genomic DNA
tccactccgctgtctcctg

SEQ ID NO: 4 - DNA, 19 bp, synthetic construct
  source 1..19: mol_type = other DNA
tccactctgctgtctcctg